SEQUENCE LISTING (20 sequences)

SEQ ID NO: 1 - protein, 9 aa, Homo sapiens
AVVWGVTAV

SEQ ID NO: 2 - protein, 9 aa, Homo sapiens
AVVTGVTAV

SEQ ID NO: 3 - protein, 12 aa, Artificial
  Synthetic peptide
GAVVWGVTAVKK

SEQ ID NO: 4 - protein, 12 aa, Artificial
  Synthetic peptide
RAVVTGVTAVAE

SEQ ID NO: 5 - protein, 15 aa, Artificial
  Synthetic peptide
GAVVWGVTAVKKKKK

SEQ ID NO: 6 - protein, 24 aa, Artificial
  Synthetic peptide
GAVVWGVTAVKKGRKKRRQRRRPQ

SEQ ID NO: 7 - protein, 22 aa, Artificial
  Synthetic peptide
YGRKKRRQRRRAVVTGVTAVAE

SEQ ID NO: 8 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 9 - DNA, 1926 bp, Artificial
  hybrid CMV enhancer/CBA promoter
agcttgcatgcctgcaggtcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttggggatcgtacctaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttg

SEQ ID NO: 10 - DNA, 587 bp, Artificial
  Woodchuck post-transcriptional regulatory sequence
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 11 - DNA, 6 bp, Bos taurus
aataaa

SEQ ID NO: 12 - DNA, 837 bp, Artificial
  scaffold attachment region sequence
agcttgcatgcctgcaggtcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttggggatc

SEQ ID NO: 13 - DNA, 6467 bp, Artificial
  S62 vector sequence
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacgatccccagcttgcatgcctgcaggtcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttggggatcgtacctaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagttagccatgggcgccgtggtgtggggcgtgaccgccgtgaagaagggcaggaagaagaggaggcagaggaggaggccccagagatcttatccgtatgatgttcctgattatgcttgatagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 14 - DNA, 6461 bp, Artificial
  S71 vector sequence
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtacgatccccagcttgcatgcctgcaggtcactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttggggatcgtacctaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagttagccatgtacggcaggaagaagaggaggcagaggaggagggccgtggtgaccggcgtgaccgccgtggccgagagatcttatccgtatgatgttcctgattatgcttgatagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 15 - protein, 12 aa, Artificial
  Synthetic peptide
GRKKRRQRRRPQ

SEQ ID NO: 16 - protein, 9 aa, Artificial
  Synthetic peptide
YPYDVPDYA

SEQ ID NO: 17 - protein, 6 aa, Artificial
  Synthetic peptide
KKKKKK

SEQ ID NO: 18 - protein, 6 aa, Artificial
  Synthetic peptide
HHHHHH

SEQ ID NO: 19 - protein, 10 aa, Homo sapiens
EQKLISEEDL

SEQ ID NO: 20 - protein, 8 aa, Artificial
  Synthetic peptide
DYKDDDDK

(19) United States
(12) Patent Application Publication
(10) Pub. No.: US 2024/0025953 A1
(43) Pub. Date: Jan. 25, 2024

(54) COMPOSITIONS AND METHODS FOR INHIBITING ALPHA-SYNUCLEIN AGGREGATION

(71) Applicant: Seelos Therapeutics, Inc., New York, NY (US)

(72) Inventors: Raj Mehra, New York, NY (US); Timothy Whitaker, New York, NY (US)

(21) Appl. No.: 18/027,250

(22) PCT Filed: Sep. 23, 2021
(86) PCT No.: PCT/US2021/051758

§ 371 Date: Mar. 20, 2023

Related U.S. Application Data
(60) Provisional application No. 63/082,208, filed on Sep. 23, 2020.

(51) Int. Cl. C07K 14/47; C12N 15/86; A61K 48/00

(52) U.S. Cl. CPC C07K 14/47; C12N 15/86; A61K 48/005; A61K 48/0075; C12N 2750/14143; C07K 2319/42; C12N 2830/15; C12N 2830/60

(57) ABSTRACT

Provided herein are methods and compositions useful for the treatment of neurodegenerative diseases that include adeno-associated viral vectors that encode one or more peptides that inhibit α-synuclein aggregation. Such peptides are useful for treating neurodegenerative diseases such as Alzheimer's disease, a Lewy body disorder, Parkinson's disease (PD), PD with dementia (PDD), pure autonomic failure (PAF), and multiple system atrophy (MSA).

### TECHNICAL FIELD

This disclosure provides methods and compositions useful for the treatment of neurodegenerative diseases. In particular, this disclosure provides methods and compositions useful for the treatment of neurodegenerative diseases such as Parkinson's disease by administering an adeno-associated viral vector that encode one or more peptides that inhibit α-synuclein aggregation.

### BACKGROUND

Synucleinopathies such as Parkinson's disease (PD), dementia with Lewy bodies (DLB) and multiple system atrophy (MSA) are a class of neurodegenerative diseases characterized by the abnormal accumulation of aggregates of α-synuclein (α-syn) protein in the cytoplasm of selective populations of neurons and glia. These aggregates are the main component of both Lewy bodies, the defining histological feature of PD and DLB, and oligodendroglial inclusions, which are found in MSA. Such aggregates have been shown to accompany neuronal damage (Spillantini et al. Nature. 1997; 388(6645):839-40 and Ubhi et al. Trends Neurosci. 2011 November; 34(11): 581-590). Deposition of aggregates of synuclein in neurons and glia suggests that a common pathogenic mechanism may exist for these disorders.

Based on structural studies, a number of different models for α-synuclein fibrils have been proposed. Limited proteolysis and NMR studies suggest that the fibril core is composed of residues 30-100 (Miake et al. J. Biol. Chem. 2002; 277(21):19213-9). Crystal structures and NMR studies suggest different models of α-syn fibrils. In one model based on crystal structures of short segments, two monomers per sheet form extended steric zippers (Rodriguez et al. Nature. 2015; 525(7570):486-490). In a second ssNMR-based model, a greek key topology with one monomer per amyloid layer has been shown (Tuttle et al. Nat. Struct. Mol. Biol. 2016; 23(5):409-415). Taken together these studies suggest that α-syn can form polymorphic fibrillar architectures.

PD is one of the most common movement disorders and DLB is the second most common cause of degenerative dementia. A causative link between α-synuclein amyloid formation and disease progression is supported by the findings that gene duplications and familial mutations that increase amyloid load also cause early-onset PD, and more than 90% of sporadic PD patients stain positive for α-synuclein deposits. Cardinal clinical features of PD include slowness of movement, decrease of amplitude and speed, as well as bradykinesia, rest tremor, and rigidity (see Urbizu and Beyer. Int J Mol Sci. 2020 July; 21(13): 4718).

There is currently no cure for neurodegenerative diseases such as PD. Current treatments focus on the relief of symptoms and improvements to the quality of life. The value of current therapies for Parkinson's disease (PD) and other neurodegenerative diseases is limited by the fact they do little, if anything, to modify the underlying progression of the disease. Therapies with disease modifying properties, e.g., therapies that are neuroprotective, neurorestorative or slow, halt or even reverse disease progression, will be of great value. As such, there is a need to identify agents which prevent and/or inhibit α-synuclein aggregation and/or cytotoxicity.

### SUMMARY

In some embodiments, provided herein are compositions that include a viral vector, wherein the viral vector includes a coding sequence that encodes a peptide that include the amino acid sequence: AVVWGVTAV (SEQ ID NO: 1) or AVVTGVTAV (SEQ ID NO: 2). In some embodiments, the composition includes a coding sequence that encodes a peptide that include the amino acid sequence: GAVVWGVTAVKK (SEQ ID NO: 3) or RAVVTGVTAVAE (SEQ ID NO: 4). In some embodiments, the composition includes a coding sequence that encodes a peptide that include the amino acid sequence: GAVVWGVTAVKKGRKKRRQRRRPQ (SEQ ID NO: 6) or YGRKKRRQRRRAVVTGVTAVAE (SEQ ID NO: 7). In some embodiments, the peptide is from 9 to 45 amino acids in length. In some embodiments, the peptide is from 30 to 40 amino acids in length. In some embodiments, the peptide is 34 amino acids in length. In some embodiments, the peptide is 36 amino acids in length. In some embodiments, compositions that include a viral vector provided herein include a viral vector that includes a coding sequence that encodes a peptide that inhibits α-synuclein (SEQ ID NO: 8) aggregation by binding to residues 68-78 of α-synuclein.

In some embodiments, the viral vector is an AAV vector. In some embodiments, the AAV vector is an AAV serotype 1 vector (AAV1), an AAV serotype 2 vector (AAV2), an AAV serotype 1 and 2 hybrid vector (AAV1/2), an AAV serotype 3 vector (AAV3), an AAV serotype 4 vector (AAV4), an AAV serotype 5 vector (AAV5), an AAV serotype 6 vector (AAV6), an AAV serotype 7 vector (AAV7), an AAV serotype 8 vector (AAV8), or an AAV serotype 9 vector (AAV9). In some embodiments, the AAV vector is an AAV1 vector, an AAV2 vector, an AAV1/2 vector, an AAV4 vector, an AAV5 vector, an AAV8 vector, or an AAV9 vector. In some embodiments, the AAV vector is an AAV1/2 vector.

In some embodiments, compositions that include a viral vector provided herein include a viral vector that includes a coding sequence that encodes a heterologous peptide tag. In some embodiments, the heterologous peptide tag is coupled to the peptide. In some embodiments, the heterologous peptide tag is a human influenza hemagglutinin (HA) tag.

In some embodiments, compositions that include a viral vector provided herein include a viral vector that includes a regulatory sequence. In some embodiments, regulatory sequence includes a promoter. In some embodiments, the promoter is an inducible promoter, a constitutive promoter, or a tissue-specific promoter. In some embodiments, the promoter is the JC polymovirus promoter, the platelet-derived growth factor B-chain (PDGF-beta) promoter, the chicken β-actin (CBA) promoter, or the cytomegalovirus (CMV) promoter. In some embodiments, the regulatory sequence is a SV40 early enhancer/promoter element, a hybrid CMV enhancer/PDGF-beta promoter element, or a hybrid CMV enhancer/CBA promoter element. In some embodiments, the regulatory sequence is a hybrid CMV enhancer/CBA promoter. In some embodiments, the hybrid CMV enhancer/CBA promoter includes a nucleic acid sequence of SEQ ID NO: 9.

In some embodiments, compositions that include a viral vector provided herein include a viral vector that includes a post-transcriptional regulatory element. In some embodiments, the post-transcriptional regulatory element is a Woodchuck post-transcriptional regulatory element. In some embodiments, the Woodchuck post-transcriptional regulatory element includes a nucleic acid sequence of SEQ ID NO: 10.

In some embodiments, compositions that include a viral vector provided herein include a viral vector that includes a scaffold attachment region sequence. In some embodiments, the scaffold attachment region sequence includes a nucleic acid sequence of SEQ ID NO: 12.

In some embodiments, compositions that include a viral vector provided herein include a viral vector that includes a polyadenylation signal sequence. In some embodiments, the polyadenylation sequence is a bovine growth hormone (BGH) polyadenylation signal sequence. In some embodiments, the bovine growth hormone (BGH) polyadenylation signal sequence includes a nucleic acid sequence of SEQ ID NO: 11.

In some embodiments, compositions that include a viral vector provided herein include a viral vector that includes a nucleic acid sequence of SEQ ID NO: 13 or SEQ ID NO: 14.

In some embodiments, compositions that include a viral vector provided herein further include a pharmaceutically acceptable excipient.

In some embodiments, provided herein are kits that include any of the compositions that include a viral vector provided herein. In some embodiments, kits provide herein further include a pre-loaded syringe that include the composition.

In some embodiments, provided herein are adeno-associated viral (AAV) vectors, wherein the AAV includes a coding sequence that encodes a peptide that include the amino acid sequence: AVVWGVTAV (SEQ ID NO: 1) or AVVTGVTAV (SEQ ID NO: 2). In some embodiments, the AAV includes a coding sequence that encodes a peptide that include the amino acid sequence: GAVVWGVTAVKK (SEQ ID NO: 3) or RAVVTGVTAVAE (SEQ ID NO: 4). In some embodiments, the AAV includes a coding sequence that encodes a peptide that include the amino acid sequence: GAVVWGVTAVKKGRKKRRQRRRPQ (SEQ ID NO: 6) or YGRKKRRQRRRAVVTGVTAVAE (SEQ ID NO: 7). In some embodiments, the peptide is from 9 to 45 amino acids in length. In some embodiments, the peptide is from 30 to 40 amino acids in length. In some embodiments, the peptide is 34 amino acids in length. In some embodiments, the peptide is 36 amino acids in length. In some embodiments, AAVs provided herein include a coding sequence that encodes a peptide that inhibits α-synuclein (SEQ ID NO: 8) aggregation by binding to residues 68-78 of α-synuclein.

In some embodiments, AAVs provided herein are an AAV serotype 1 vector (AAV1), an AAV serotype 2 vector (AAV2), an AAV serotype 1 and 2 hybrid vector (AAV1/2), an AAV serotype 3 vector (AAV3), an AAV serotype 4 vector (AAV4), an AAV serotype 5 vector (AAV5), an AAV serotype 6 vector (AAV6), an AAV serotype 7 vector (AAV7), an AAV serotype 8 vector (AAV8), or an AAV serotype 9 vector (AAV9). In some embodiments, AAVs provided herein are an AAV1 vector, an AAV2 vector, an AAV1/2 vector, an AAV4 vector, an AAV5 vector, an AAV8 vector, or an AAV9 vector. In some embodiments, the AAV vector is an AAV1/2 vector.

In some embodiments, AAVs provided herein include a coding sequence that encodes a heterologous peptide tag. In some embodiments, the heterologous peptide tag is coupled to the peptide. In some embodiments, the heterologous peptide tag is a human influenza hemagglutinin (HA) tag.

In some embodiments, the AAV vector includes a regulatory sequence. In some embodiments, the regulatory sequence includes a promoter. In some embodiments, the promoter is an inducible promoter, a constitutive promoter, or a tissue-specific promoter. In some embodiments, the promoter is the JC polymovirus promoter, the platelet-derived growth factor B-chain (PDGF-beta) promoter, the chicken β-actin (CBA) promoter, or the cytomegalovirus (CMV) promoter. In some embodiments, the regulatory sequence is the SV40 early enhancer/promoter element, the hybrid CMV enhancer/PDGF-beta promoter element, or the hybrid CMV enhancer/CBA promoter element. In some embodiments, the regulatory sequence is the hybrid CMV enhancer/CBA promoter. In some embodiments, the hybrid CMV enhancer/CBA promoter includes a nucleic acid sequence of SEQ ID NO: 9.

In some embodiments, the AAV vector includes a post-transcriptional regulatory element. In some embodiments, the post-transcriptional regulatory element is a Woodchuck post-transcriptional regulatory element. In some embodiments, the Woodchuck post-transcriptional regulatory element includes a nucleic acid sequence of SEQ ID NO: 10.

In some embodiments, the AAV vector includes a scaffold attachment region sequence. In some embodiments, the scaffold attachment region sequence includes a nucleic acid sequence of SEQ ID NO: 12.

In some embodiments, the AAV vector includes a polyadenylation sequence. In some embodiments, the polyadenylation sequence is a bovine growth hormone (BGH) polyadenylation sequence. In some embodiments, the bovine growth hormone (BGH) polyadenylation sequence includes a nucleic acid sequence of SEQ ID NO: 11.

In some embodiments, the AAV vector includes a nucleic acid sequence of SEQ ID NO: 13 or SEQ ID NO: 14.

In some embodiments, provided herein are methods for treating a neurodegenerative disease in a subject, the method that include administering to the subject an effective amount of any of the compositions or any of the AAV vectors provided herein. In some embodiments, the neurodegenerative disease is Alzheimer's disease or a synucleinopathy. In some embodiments, the synucleinopathy is selected from the group consisting of: a Lewy body disorder, Parkinson's disease (PD), PD with dementia (PDD), pure autonomic failure (PAF), and multiple system atrophy (MSA).

In some embodiments, provided herein are methods for reducing α-synuclein (SEQ ID NO: 8) aggregation in a subject in need thereof, the method that include administering to the subject an effective amount of any of the compositions or any of the AAV vectors provided herein. In some embodiments, the neurodegenerative disease is Alzheimer's disease or a synucleinopathy. In some embodiments, the synucleinopathy is selected from the group consisting of: a Lewy body disorder, Parkinson's disease (PD), PD with dementia (PDD), pure autonomic failure (PAF), and multiple system atrophy (MSA).

In some embodiments of any of the methods provided herein, the composition or the AAV vector is administered parenterally. In some embodiments, the composition or the AAV vector is administered to the central nervous system of the subject. In some embodiments, the composition or the AAV vector is administered intracranially. In some embodiments, the composition or the AAV vector is administered to the substantia nigra of the subject.

In some embodiments, provided herein are methods of expressing a peptide that inhibits α-synuclein (SEQ ID NO: 8) aggregation in a mammalian cell, the method that include introducing any of the AAV vectors provided herein into the mammalian cell. In some embodiments, the mammalian cell is a neuron or neuroglial cell. In some embodiments, the mammalian cell is a neuron.

In some embodiments, provided herein are kits that include any of the compositions or any of the AAV vectors provided herein, and instructions for performing any of the methods provided herein.

The term “amino acid” refers to any amino acid, including naturally occurring amino acids (e.g., alpha-amino acids), unnatural amino acids, and modified amino acids. The term “amino acid” includes both D- and L-amino acids. Non-limiting examples of unnatural amino acids include beta-amino acids, homo-amino acids, proline and pyruvic acid derivatives, 3-substituted alanine derivatives, glycine derivatives, ring substituted phenylalanine and tyrosine derivatives, linear core amino acids, and N-methyl amino acids. A modified amino acid can be an amino acid resulting from a reaction at an amino group, carboxy group, side-chain functional group, or from the replacement of any hydrogen by a heteroatom. Amino acids are referred to herein by their full name and/or by their IUPAC one-letter abbreviation.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients.

The details of one or more embodiments of the invention are set forth in the accompanying drawings and the description below. Other features, objects, and advantages of the invention will be apparent from the description and drawings, and from the claims. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DETAILED DESCRIPTION

This disclosure provides viral vectors that encode one or more peptides that inhibit α-synuclein aggregation. Also provided herein are methods of inhibiting α-synuclein aggregation in a subject using viral vectors that encode one or more peptides that inhibit α-synuclein aggregation. The α-synuclein protein, a 140 amino acid protein (SEQ ID NO: 8) that has been found in amyloid deposits in neuronal cells in disease conditions, plays a fundamental role in the pathogenesis of synucleinopathies such as Parkinson's disease, dementia with Lewy bodies, and multiple system atrophy (see, e.g., Visanji et al. Transl Neurodegener. 2019; 8:28). Abnormal deposition of α-synuclein is also observed in a variety of other diseases including Alzheimer's disease, pure autonomic failure, and REM sleep behavior disorder (Brás et al. J Neurochem. 2020; 153(4):433-454). Accordingly, the methods described herein are useful, for example, for treating a condition or disease in which α-synuclein aggregation contributes to the pathology and/or symptoms and/or progression of the condition or disease (e.g., a neurodegenerative disease) in a subject (e.g., a human). This disclosure also provides compositions containing the viral vectors described herein and methods to express the disclosed peptide inhibitors in, e.g., a subject in need thereof, using a viral vector that encodes one or more peptides that inhibit α-synuclein aggregation.

A “peptide that inhibits α-synuclein aggregation” or a “peptide inhibitor of α-synuclein aggregation” is any peptide exhibiting one or more of the following activities: decreasing the rate of α-synuclein aggregation, decreasing the amount of α-synuclein aggregation, decreasing the spread of α-synuclein aggregation, preventing α-synuclein aggregation, stopping α-synuclein aggregation, eliminating α-synuclein aggregation reducing and/or eliminating the toxicity of α-synuclein aggregates, and reducing and/or eliminating the spread of α-synuclein aggregates.

Seeding, the sequential transfer of pathologic protein aggregates along connected tissues, and/or prion-like spread of α-synuclein aggregates can contribute to the progression and severity of neurodegenerative diseases. For example, Braak staging has shown that pathology gradually spreads over time through connected brain regions, and cell culture and animal models show that small amounts of α-syn aggregates can act as seeds and induce aggregation of the native protein (see, e.g. Braak H, et al. (2003) Neurobiol Aging 24(2): 197-211; Braak et al. (2009) Adv Anat Embryol Cell Biol 201: 1-119; Masuda-Suzukake M, et al. (2013) Brain 136(4): 1128-1138; Desplats P, et al. (2009) Proc Natl Acad Sci USA 106(31): 13010-13015; Luk K C, et al. (2009) Proc Natl Acad Sci 106(47):20051-20056). In some embodiments, a peptide expressed by a viral vector provided herein inhibits aggregation of α-synuclein. For example, a peptide that inhibits aggregation of α-synuclein can inhibit α-synuclein aggregation by about 10%, by about 20%, by about 30%, by about 40%, by about 50%, by about 60%, by about 70%, by about 80%, by about 90%, by about 95%, by about 96%, by about 97%, by about 98%, by about 99%, or more as compared to the α-synuclein aggregation that would be observed in the absence of the peptide. In some embodiments, a peptide that inhibits α-synuclein aggregation “caps” growing aggregates of α-synuclein, preventing the spread of α-synuclein aggregates.

Cell-based assays can be used to test the efficiency with which peptides can prevent seeding and/or cap growing aggregates of α-synuclein. For example, endogenous protein aggregation or puncta formation can be measured after transfection of nanomolar amounts of α-synuclein seeds in cell, e.g., HEK293 cells, and administration of a peptide that inhibits α-synuclein aggregation. In some embodiments, the cell can express fluorescently-labeled (e.g., YFP-labeled) α-synuclein and aggregation can be monitored by fluorescent imaging (see, e.g., Sanders D W, et al. (2014) Neuron 82(6): 1271-1288 and U.S. Application No. 2019/0241613). In vitro aggregation assays can also be used to test the efficacy of a peptide that inhibit α-synuclein aggregation. For example, recombinantly purified α-synuclein can be aggregated in the presence of a peptide, and the aggregation can be monitored by measuring fluorescence of Thioflavin T, an amyloid binding dye. In some embodiments, insoluble protein aggregates extracted from tissue samples (e.g., tissue samples from PD patients) can be used to seed α-synuclein aggregation in in vitro and in vivo models.

In some embodiments, the peptide that inhibits α-synuclein aggregation includes an amino acid sequence of AVVWGVTAV (SEQ ID NO: 1). In some embodiments, the peptide that inhibits α-synuclein aggregation is AVVWGVTAV (SEQ ID NO: 1). In some embodiments, the peptide that inhibits α-synuclein aggregation includes an amino acid sequence of AVVTGVTAV (SEQ ID NO: 2). In some embodiments, the peptide that inhibits α-synuclein aggregation is AVVTGVTAV (SEQ ID NO: 2). Peptides that are or include an amino acid sequence of SEQ ID NO:1 or SEQ ID NO:2 have been shown to prevent formation of seeding competent aggregates as well as cap fibril seeds and prevent their elongation (see, e.g., U.S. Application No. 2019/0241613).

In some embodiments, the peptide that inhibits α-synuclein aggregation binds to the NACore (residues 68-78) of α-synuclein. The NACore is within the 35-residue NAC (non-amyloid 3 component) domain found in amyloid deposits (Rodriguez J A, et al. (2015) Nature 525(7570):486-490). The NACore aggregates readily, and the aggregates display properties such as diffraction pattern and cytotoxicity similar to full-length α-syn (see, e.g., U.S. Application No. 2019/0241613). In some embodiments, the peptide encoded by a viral vector described herein inhibits α-synuclein aggregation by binding to residues 68-78 of α-synuclein. Residues 47-56 of α-synuclein are referred to as “PreNAC” and may also lead to the formation of aggregates.

In some embodiments, a peptide that inhibits α-synuclein aggregation is from about 9 to about 45 amino acids in length. For example, a peptide that inhibits α-synuclein aggregation can be from about 9 to about 15, about 9 to about 20, about 9 to about 25, about 9 to about 30, about 9 to about 35, about 9 to about 40, about 40 to about 45, about 35 to about 45, about 30 to about 45, about 25 to about 45, about 20 to about 45, or about 15 to about 45 amino acids in length. In some embodiments, the peptide is from about 25 to about 35, about 30 to about 40, or about 35 to about 45 amino acids in length. For example, a peptide that inhibits α-synuclein aggregation can be 31, 32, 33, 34, 35, 36, 37, 38, 39, or 40 amino acids in length. In some embodiments, the peptide that inhibits α-synuclein aggregation is 34 amino acids in length. In some embodiments, the peptide that inhibits α-synuclein aggregation is 36 amino acids in length.

In some embodiments, a peptide that inhibits α-synuclein aggregation includes a heterologous peptide tag. In some embodiments, can help prevent self-aggregation of the peptide, increase solubility of the peptide, facilitate monitoring of the peptide, or a combination thereof. Non-limiting examples of such heterologous peptide tags include a TAT tag, a human influenza hemagglutinin (HA) tag, a polyLys tag, a GST tag, a GFP tag, a polyHis tag, a V5 tag, a Myc tag, and a FLAG tag. See the exemplary sequences of exemplary heterologous peptide tags in Table 1. In some embodiments, the heterologous peptide tag is an HA tag.

TABLE 1

| Tag | SEQ ID NO | Sequence |
|---|---|---|
| TAT | 15 | GRKKRRQRRRPQ |
| HA | 16 | YPYDVPDYA |
| polyLys | 17 | KKKKKK |
| polyHis | 18 | HHHHHH |
| V5 | 19 | GKPIPNPLLGLDST |
| Myc | 20 | EQKLISEEDL |
| FLAG | 21 | DYKDDDDK |

In some embodiments, the heterologous peptide tag is at the N-terminus of the peptide that inhibits α-synuclein aggregation. In some embodiments, the heterologous peptide tag is at the C-terminus of the peptide that inhibits α-synuclein aggregation.

In some embodiments, a peptide that inhibits α-synuclein aggregation includes the amino acid sequence: AVVWGVTAV (SEQ ID NO: 1) or GAVVWGVTAVKK (SEQ ID NO: 3). In some embodiments, a peptide that inhibits α-synuclein aggregation is AVVWGVTAV (SEQ ID NO: 1) or GAVVWGVTAVKK (SEQ ID NO: 3). In some embodiments, a peptide that inhibits α-synuclein aggregation includes the amino acid sequence: AVVTGVTAV (SEQ ID NO: 2) or RAVVTGVTAVAE (SEQ ID NO: 4). In some embodiments, a peptide that inhibits α-synuclein aggregation is AVVTGVTAV (SEQ ID NO: 2) or RAVVTGVTAVAE (SEQ ID NO: 4). In some embodiments, the peptide that inhibits α-synuclein aggregation includes one or more heterologous peptide tags (e.g., any of the heterologous peptide tags in Table 1) at either N-terminal to or C-terminal to the peptide that inhibits α-synuclein aggregation. In some embodiments, the peptide that inhibits α-synuclein aggregation includes a polyLys tag. For example, in some embodiments, the peptide that inhibits α-synuclein aggregation includes the amino acid sequence: GAVVWGVTAVKKKKK (SEQ ID NO: 5). In some embodiments, the peptide that inhibits α-synuclein aggregation is GAVVWGVTAVKKKKK (SEQ ID NO: 5). In some embodiments, the peptide that inhibits α-synuclein aggregation has a TAT tag. For example, in some embodiments, the peptide that inhibits α-synuclein aggregation includes the amino acid sequence: GAVVWGVTAVKKGRKKRRQRRRPQ (SEQ ID NO: 6). In some embodiments, the peptide that inhibits α-synuclein aggregation is GAVVWGVTAVKKGRKKRRQRRRPQ (SEQ ID NO: 6). In some embodiments, the peptide that inhibits α-synuclein aggregation includes the amino acid sequence: YGRKKRRQRRRAVVTGVTAVAE (SEQ ID NO: 7). In some embodiments, the peptide that inhibits α-synuclein aggregation is YGRKKRRQRRRAVVTGVTAVAE (SEQ ID NO: 7).

In some embodiments, a peptide that inhibits α-synuclein aggregation includes two or more monomers separated by peptide linkers. For example, a peptide that inhibits α-synuclein aggregation can include two, three, four, or more monomers separated by peptide linkers. In some embodiments, a peptide that inhibits α-synuclein aggregation includes four monomers separated by peptide linkers. In some embodiments, a peptide that inhibits α-synuclein aggregation includes two or more monomers separated by peptide linkers wherein the peptide monomers include the sequence AVVWGVTAV (SEQ ID NO: 1). In some embodiments, a peptide that inhibits α-synuclein aggregation includes two or more monomers separated by peptide linkers wherein the peptide monomers include the sequence GAVVWGVTAVKK (SEQ ID NO: 3). In some embodiments, a peptide that inhibits α-synuclein aggregation includes two or more monomers separated by peptide linkers wherein the peptide monomers include the sequence GAVVWGVTAVKKKKK (SEQ ID NO: 5). In some embodiments, a peptide that inhibits α-synuclein aggregation includes two or more monomers separated by peptide linkers wherein the peptide monomers include the sequence GAVVWGVTAVKKGRKKRRQRRRPQ (SEQ ID NO: 6). In some embodiments, a peptide that inhibits α-synuclein aggregation includes four identical monomers selected from the group consisting of: AVVWGVTAV (SEQ ID NO: 1), GAVVWGVTAVKK (SEQ ID NO: 3), GAVVWGVTAVKKKKK (SEQ ID NO: 5), and GAVVWGVTAVKKGRKKRRQRRRPQ (SEQ ID NO: 6). In some embodiments, a peptide that inhibits α-synuclein aggregation includes four monomers separated by peptide linkers wherein the peptide monomers are individually selected from monomers that include the sequence AVVWGVTAV (SEQ ID NO: 1), GAVVWGVTAVKK (SEQ ID NO: 3), GAVVWGVTAVKKKKK (SEQ ID NO: 5), or GAVVWGVTAVKKGRKKRRQRRRPQ (SEQ ID NO: 6).

In some embodiments, a peptide that inhibits α-synuclein aggregation includes two or more monomers separated by peptide linkers, wherein the linkers are encoded by nucleic acids having sequences selected from the following group: gggggaggtggctctggtggcggagggtca (SEQ ID NO: 22), ggaggcggtgggagcggtgggggaggtagt (SEQ ID NO: 23), ggtggggggggaagtggagggggtggctct (SEQ ID NO: 24), ggtggtggaggatctgggggcggtggttct (SEQ ID NO: 25), and combinations thereof. In some embodiments, a peptide that inhibits α-synuclein aggregation includes two or more monomers separated by peptide linkers, wherein the peptide linkers are all identical. In some embodiments, a peptide that inhibits α-synuclein aggregation includes two or more monomers separated by peptide linkers, wherein two, three, or four of the peptide linkers are identical. In some embodiments, a peptide that inhibits α-synuclein aggregation includes two or more monomers separated by peptide linkers, wherein the peptide linkers are not all identical.

### Viral Vectors

Provided herein are viral vectors that encode one or more peptides that inhibit α-synuclein aggregation (e.g., any of the peptides described herein). The term “viral vector” as referred to herein includes any viral genetic element that is capable of replication when associated with the proper control elements and that can transfer gene sequences between cells. In some embodiments, useful vectors are contemplated to be those vectors in which the nucleic acid segment to be transcribed is positioned under the transcriptional control of a promoter. A viral vector can include, for example, sufficient cis-acting elements for expression; other elements for expression can be supplied by the host mammalian cell or in an in vitro expression system. Viral vectors include all those known in the art, such as, without limitation, adeno-associated viral (AAV) vectors, retroviral vectors, Herpes simplex vectors, alphavirus vectors, flavivirus vectors, rhabdovirus-based vectors, and chimeric viral vectors.

In some embodiments, the viral vector is an AAV vector (see, e.g., Asokan et al., Mol. Ther. 20: 699-7080, 2012). “Recombinant AAV vectors” or “rAAVs” are typically composed of, at a minimum, a transgene or a portion thereof and a regulatory sequence, and optionally 5′ and 3′ AAV inverted terminal repeats (ITRs). Such a recombinant AAV vector can be packaged into a capsid and delivered to a selected target cell (e.g., a neuronal or neuroglial cell). The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 to about 210 nucleotides in length. In some embodiments, substantially the entire sequences encoding the ITRs are used in the molecule, although some degree of minor modification of these sequences is permissible. The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

Non-limiting examples of an AAV vector include an AAV serotype 1 vector (AAV1), an AAV serotype 2 vector (AAV2), an AAV serotype 1 and 2 hybrid vector (AAV1/2), an AAV serotype 3 vector (AAV3), an AAV serotype 4 vector (AAV4), an AAV serotype 5 vector (AAV5), an AAV serotype 6 vector (AAV6), an AAV serotype 7 vector (AAV7), an AAV serotype 8 vector (AAV8), or an AAV serotype 9 vector (AAV9). In some embodiments, the AAV vector is an AAV1 vector, an AAV2 vector, an AAV1/2 vector, an AAV4 vector, an AAV5 vector, an AAV8 vector, or an AAV9 vector. In some embodiments, the AAV vector is an AAV1/2 vector.

In some embodiments, the viral vector (e.g., AAV vector) has a total number of nucleotides of up to about 10 kb. In some embodiments, the viral vector has a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb. In some embodiments, the viral vector has a total number of nucleotides of about 6kb to about 7 kb. In some embodiments, the viral vector has a total number of nucleotides of about 6.5 kb.

In some embodiments, a viral vector (e.g., AAV vector) as described herein also includes one or more regulatory sequences that are operably linked to the transgene (e.g., a nucleic acid encoding a peptide that inhibits α-synuclein aggregation, e.g., any of the peptides described herein) in a manner which permits its transcription, translation, and/or expression in a cell transfected with the viral vector or infected with a virus containing the viral vector. The term “regulatory sequence” refers to a nucleic acid sequence that regulates expression of a gene product operably linked to the regulatory sequence. The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Such 5′ non-transcribed regulatory sequences can include a promoter region that includes a promoter sequence for transcriptional control of the operably joined gene. The viral vectors described herein can also include regulatory sequences such as enhancer sequences, hybrid enhancer/promoter sequences, and upstream activator sequences as desired. See, e.g., Powell et al. Discov Med. 2015; 19(102): 49-57 and Hagedorn et al. Hum Gene Ther. 2017; 28(12):1169-1179. The viral vectors described herein can optionally include 5′ leader or signal sequences. The choice and design of an appropriate vector is within the ability and discretion of one of ordinary skill in the art.

As used herein, a nucleic acid sequence (e.g., coding sequence) and a regulatory sequence are said to be “operably” linked when they are covalently linked in such a way as to place the expression or transcription of the nucleic acid sequence under the influence or control of the regulatory sequences. If it is desired that the nucleic acid sequence be translated into a functional protein or peptide, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame.

The term “promoter” refers to a nucleic acid sequence that is operably linked to a nucleic acid sequence encoding a polypeptide (e.g., a peptide described herein, e.g., a peptide that inhibits α-synuclein aggregation) that can increase the transcription of the nucleic acid sequence encoding the polypeptide. The phrases “operatively positioned,” “under control” or “under transcriptional control” means that the promoter is in the correct location and orientation in relation to the nucleic acid to control RNA polymerase initiation and expression of the gene.

In some embodiments, a promoter is constitutive. Non-limiting examples of constitutive promoters include the retroviral Rous sarcoma virus (RSV) LTR promoter (optionally with the RSV enhancer), the cytomegalovirus (CMV) promoter (optionally with the CMV enhancer) (see, e.g., Boshart et al. Cell. 1985; 41:521-530), the SV40 promoter, the dihydrofolate reductase promoter, the R-actin promoter, the phosphoglycerol kinase (PGK) promoter, and the EF1α, promoter (Invitrogen).

In some embodiments, a promoter is inducible. Inducible promoters allow regulation of gene expression and can be regulated by a variety of conditions including, but not limited to, exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Non-limiting examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al. Proc. Natl. Acad. Sci. USA, 1996; 93:3346-3351), the tetracycline-repressible system (Gossen et al. Proc. Natl. Acad. Sci. USA, 1995; 89:5547-5551), the tetracycline-inducible system (Gossen et al. Science, 1995; 268: 1766-1769), see also Harvey et al. Curr. Opin. Chem. Biol., 1998; 2:512-518), the RU486-inducible system (Wang et al. Nat. Biotech., 1997; 15:239-243 and Wang et al. Gene Then, 1997; 4:432-441) and the rapamycin-inducible system (Magari et al. J. Clin. Invest. 1997; 100:2865-2872). Certain types of inducible promoters which can be useful in this context are those that are regulated by a specific physiological state, e.g., temperature, acute phase, a particular differentiation state of the cell, or in replicating cells only.

In some embodiments, a regulatory sequence imparts tissue-specific gene expression capabilities. Such tissue-specific regulatory sequences (e.g., promoters, enhancers, etc.) are well known in the art. In some embodiments, the tissue-specific regulatory sequence is a tissue-specific promoter. The term “tissue-specific” promoter refers to a nucleotide sequence which, when operably linked to a polynucleotide that encodes a gene product, causes the gene product to be produced in a cell substantially only if the cell is a cell of the tissue type corresponding to the promoter. In some embodiments, a tissue-specific promoter binds a tissue-specific transcription factor that induces transcription in a tissue-specific manner. Non-limiting examples of tissue-specific promoters include: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter.

Other exemplary promoters that can be used in accordance with materials and methods provided herein include 0-glucuronidase (GUSB) promoter, GUSB minimal promoter (hGBp), methyl CpG-Binding Protein-2 (MECP2) promoter, human desmin (DES) promoter, human thyroxine binding globulin (TBG) promoter, promoter of HNRPA2B1-CBX3 (UCOE), muscle creatine kinase (MCK) promoter, synthetic muscle promoter C5-12, human alpha(1) antitrypsin (hAAT) promoters, human EFla promoter, human cytomegalovirus (CMV) promoter (U.S. Pat. No. 5,168,062), human ubiquitin C (UBC) promoter, mouse phosphoglycerate kinase 1 promoter, polyoma adenovirus promoter, simian virus 40 (SV40) promoter, β-globin promoter, β-actin promoter, γ-globin promoter, β-interferon promoter, γ-glutamyl transferase promoter, mouse mammary tumor virus (MMTV) promoter, Rous sarcoma virus promoter, rat insulin promoter, glyceraldehyde-3-phosphate dehydrogenase promoter, metallothionein II (MT II) promoter, amylase promoter, cathepsin, MI muscarinic receptor promoter, retroviral LTR (e.g. human T-cell leukemia virus HTLV) promoter, AAV ITR promoter, interleukin-2 promoter, collagenase promoter, platelet-derived growth factor promoter, adenovirus 5 E2 promoter, stromelysin, murine MX gene promoter, glucose regulated proteins (GRP78 and GRP94) promoter, α-2-macroglobulin promoter, vimentin promoter, MHC class I gene H-2κ b promoter, HSP70 promoter, proliferin, tumor necrosis factor promoter, thyroid stimulating hormone a gene promoter, immunoglobulin light chain promoter, T-cell receptor promoter, HLA DQa and DQP promoters, interleukin-2 receptor promoter, MHC class II promoter, MHC class II HLA-DRa promoter, muscle creatine kinase promoter, prealbumin (transthyretin) promoter, elastase I promoter, albumin gene promoter, c-fos promoter, c-HA-ras promoter, neural cell adhesion molecule (NCAM) promoter, H2B (TH2B) histone promoter, rat growth hormone promoter, human serum amyloid (SAA) promoter, troponin I (TN I) promoter, duchenne muscular dystrophy promoter, human immunodeficiency virus promoter, Gibbon Ape Leukemia Virus (GALV) promoters, bone osteocalcin promoter, bone sialoprotein promoter, hepatitis B virus core promoter, CD2 promoter, immunoglobulin heavy chain promoter; T cell receptor α-chain promoter, non-neuronal glial fibrillary acidic protein (GFAP) promoter, myelin basic protein (MBP) promoter, and neuronal promoters such as hSyn, neuron-specific enolase (NSE) promoter, neurofilament light-chain gene promoter, the neuron-specific vgf gene promoter, mGluR2 promoter, NFL promoter, NFH promoter, nβ2 promoter, PPE promoter, Enk promoter, excitatory amino acid transporter-2 (EAAT2) promoter, and CaMKII promoter. See, e.g., Sandig et al., Gene Then, 3:1002-9 (1996); Arbuthnot et al., Hum. Gene Ther., 7: 1503-14 (1996); Stein et al., Mol. Biol. Rep., 24: 185-96 (1997); Chen et al., J. Bone Miner. Res., 11:654-64 (1996); Hansal et al., J. Immunol., 161:1063-8 (1998); Andersen et al., Cell. Mol. Neurobiol., 13:503-15 (1993); Piccioli et al., Proc. Natl. Acad. Sci. USA, 88:5611-5 (1991); Piccioli et al., Neuron, 15:373-84 (1995); and Lodish, Molecular Cell Biology, Freeman and Company, New York 2007.

Further non-limiting examples of promoters include the JC polymovirus promoter, the platelet-derived growth factor B-chain (PDGF-beta) promoter, the chicken β-actin (CBA) promoter, or the cytomegalovirus (CMV) promoter (see, e.g., Doll et al. Gene Ther. 1996; 3(5):437-47). Additional examples of promoters are known in the art.

The term “enhancer” refers to refers to a nucleotide sequence that can increase the level of transcription of a nucleic acid encoding a protein of interest (e.g., a peptide that inhibits α-synuclein aggregation). Enhancer sequences are typically 50-1500 basepairs in length, and generally increase the level of transcription by providing additional binding sites for transcription-associated proteins (e.g., transcription factors). In some embodiments, an enhancer sequence is found within an intronic sequence. Enhancer sequences can typically act at much larger distance from the transcription start site (e.g., as compared to a promoter) than can promoters. Non-limiting examples of enhancers include a RSV enhancer, a CMV enhancer, and a SV40 enhancer.

In some embodiments, the regulatory sequence is a SV40 early enhancer/promoter, a hybrid CMV enhancer/PDGF-beta promoter, or a hybrid CMV enhancer/CBA promoter. In some embodiments, the regulatory sequence is a hybrid CMV enhancer/CBA promoter having or including a nucleic acid sequence of SEQ ID NO: 9.

In some embodiments, the viral vector includes a post-transcriptional regulatory sequence. Non-limiting examples of a post-transcriptional regulatory sequence include a Hepatitis B Virus post-transcriptional regulatory sequence and a Woodchuck post-transcriptional regulatory sequence. In some embodiments, the Woodchuck post-transcriptional regulatory sequence has or includes a nucleic acid sequence of SEQ ID NO. 10.

In some embodiments, any of the viral vectors provided herein can include a polyadenylation (poly(A)) sequence. “Polyadenylation” refers herein to the covalent linkage of a polyadenyl moiety, or its modified variant, to a messenger RNA molecule.

In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation can occur in the nucleus immediately after transcription of DNA into RNA, but it can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “polyadenylation signal sequence” or “poly(A) signal sequence” is a sequence that triggers the endonuclease cleavage of an mRNA and the addition of a series of adenosines to the 3′ end of the cleaved mRNA. For AAV vectors that include transgene nucleic acids encoding proteins, a polyadenylation sequence generally is inserted following the transgene sequences and before the 3′ AAV ITR sequence. In some embodiments, the poly(A) signal sequence is positioned 3′ to the nucleic acid sequence encoding the C-terminus of the peptide that inhibit α-synuclein aggregation.

There are several poly(A) signal sequences that can be used in the vectors described herein including those derived from bovine growth hormone (bgh) (Woychik et al., Proc. Natl. Acad. Sci. U.S.A. 81(13):3944-3948, 1984; U.S. Pat. No. 5,122,458), human growth hormone (hGH), mouse-p-globin, mouse-a-globin (Orkin et al., EMBO J. 4(2):453-456, 1985; Thein et al., Blood 7 \(2):3 \3-3 \9, 1988), human collagen, polyoma virus (Batt et al., Mol. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15(7): 1340-1347, 2007), the group consisting of SV40 poly(A) signal sequence, such as the SV40 late and early poly(A) signal sequence (Schek et al., Mol. Cell Biol. 12(12):5386-5393, 1992). In some embodiments, the poly(A) signal sequence is the sequence AATAAA. The AATAAA sequence can be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, A AT A AC, AATAGA, AATTAA, or AATAAG (see, e.g., WO 06/12414). In some embodiments, the bovine growth hormone (BGH) polyadenylation sequence has or includes a nucleic acid sequence of SEQ ID NO: 11.

In some embodiments, the poly(A) signal sequence can be a synthetic polyadenylation site (see, e.g., the pCl-neo expression vector of Promega which is based on Levitt el al, Genes Dev. 3(7): 1019-1025, 1989). In some embodiments, the poly(A) signal sequence is the polyadenylation signal of soluble neuropilin-1 (s RP) (AAATAAAATACGAAATG) (see, e.g., WO 05/073384). Additional examples of poly(A) signal sequences are known in the art.

In some embodiments, the vectors described herein can further include an upstream enhancer element (USE) that is placed upstream of a poly(A) signal sequence. Non-limiting examples of such USEs include: human immunodeficiency virus 1 (HIV-1) USE, SV40 late 2×USE, ground squirrel hepatitis virus (GHV) USE, Adenovirus (L3) USE, human prothrombin (hTHGB) USE, and human C2 complement gene (hC2) USE.

An AAV construct useful in the present disclosure can also contain an intron, e.g., located between the promoter/enhancer sequence and the transgene. See, e.g., Powell et al. Discov Med. 2015; 19(102): 49-57.

In some embodiments, any of the viral vectors provided herein can include a scaffold attachment region (SAR). As used herein, a “scaffold attachment region” refers to an AT-rich DNA sequence that binds specifically to one or more components of the nuclear scaffold. See Boulikas, J. Cell. Biochem. 52:14 (1993).

In some embodiments, the SAR has or includes a nucleic acid sequence of SEQ ID NO: 12.

Another vector element that may be used is an internal ribosome entry site (IRES). An IRES sequence is used to produce more than one polypeptide from a single gene transcript. An IRES sequence would be used to produce a protein that contains more than one polypeptide chain. Selection of these and other common vector elements is conventional, and many such sequences are available (see, e.g., Sambrook et al. “Molecular Cloning. A Laboratory Manual”, 2d ed., Cold Spring Harbor Laboratory, New York (1989), and references cited therein at, for example, pages 3.18 3.26 and 16.17 16.27 and Ausubel et al., Current Protocols in Molecular Biology, John Wiley & Sons, New York, 1989).

In some embodiments, the viral vector has or includes a nucleic acid sequence of SEQ ID NO: 13 or SEQ ID NO:14.

### Compositions

Also provided herein are compositions including a viral vector described herein (e.g., an AAV vector that encodes one or more peptides that inhibit α-synuclein aggregation). In some embodiments, the viral vector (e.g., AAV vector) is capable of expressing an α-synuclein peptide inhibitor messenger RNA in a target cell of a human subject to whom the therapeutic composition is administered. In some embodiments, the viral vector (e.g., an AAV vector capable of expressing an α-synuclein aggregation peptide inhibitor) is formulated in a pharmaceutical composition. In some embodiments, the pharmaceutical composition can include one or a plurality of AAV vectors, as described herein, in combination with one or more pharmaceutically acceptable carriers, diluents or excipients.

The phrase “pharmaceutically acceptable” indicates that the substance or composition must be compatible chemically and/or toxicologically, with the other ingredients comprising a formulation, and/or the mammal being treated therewith.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Non-limiting examples of pharmaceutically acceptable carriers that can be used in the compositions described herein include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. One of skill in the art will recognize that other pharmaceutical carriers are useful in the present disclosure.

In some embodiments, a composition described herein can also include buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; adjuvants (e.g., aluminum hydroxide); and preservatives.

In some embodiments, any of the viral vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif), formulations from Minis Bio (Madison, Wis.) and Roche Madison (Madison, Wis.), PhaseRX polymer formulations such as, without limitation, SMARTT POLYMER TECHNOLOGY® (PhaseRX, Seattle, Wash), DMRI/DOPE, poloxamer, VAXFECTIN® adjuvant from Vical (San Diego, Calif), chitosan, cyclodextrin from Calando Pharmaceuticals (Pasadena, Calif), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19: 125-132, 2008; Rozema et al., Proc. Natl. Acad. Sci. U.S.A. 104: 12982-12887, 2007; Rozema et al., Proc. Natl. Acad. Sci. U.S.A. 104: 12982-12887, 2007; Hu-Lieskovan et al., Cancer Res. 65:8984-8982, 2005; Heidel et al., Proc. Natl. Acad. Sci. U.S.A. 104:5715-5721, 2007).

Supplementary active compounds can also be incorporated into any of the compositions described herein.

Upon formulation, solutions can be administered in a manner compatible with the dosage formulation and in such amount as is therapeutically effective. The formulations can be easily administered in a variety of dosage forms such as injectable solutions, injectable gels, drug-release capsules, and the like.

In some embodiments, a composition described herein is formulated for parenteral administration. In some embodiments, a composition described herein is formulated for intracranial administration. In some embodiments, a composition described herein is formulated for intravenous administration. See, e.g., Lykken et al. J Neurodev Disord. 2018; 10(1):16.

The pharmaceutical formulations suitable for injectable use include, without limitation, sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations may contain a preservative to prevent the growth of microorganisms. In many cases, the formulation is sterile and fluid to the extent that easy syringability exists. In some embodiments, the formulation is stable under the conditions of manufacture and storage and is preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride, can be included. Prolonged absorption of the injectable compositions can be brought about by the use in the compositions of agents delaying absorption, for example, aluminum monostearate and gelatin.

For administration of an injectable aqueous solution, the solution can be suitably buffered, if necessary, and the liquid diluent can be first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage can be accommodated depending on the condition of the host. The person responsible for administration will, in any event, determine the appropriate dose for the individual host.

Sterile injectable solutions can be prepared by incorporating the active viral vector (e.g., AAV vector) in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein can also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, vesicles, and the like, can be used for the introduction of the compositions of the present invention into suitable host cells. In some embodiments, a viral vector described herein can be formulated for delivery either encapsulated in a lipid particle, a liposome, a vesicle, a nanosphere, or a nanoparticle or the like. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed. Liposomes are formed from phospholipids that are dispersed in an aqueous medium and spontaneously form multilamellar concentric bilayer vesicles (also termed multilamellar vesicles (MLVs).

### Kits

Also provided herein are kits including any of the compositions described herein. In some embodiments, a kit can include a solid composition (e.g., a lyophilized composition including a vector described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Methods

Also provided herein is are methods of introducing into cell (e.g., a neuronal or neuroglial cell) of a mammal (e.g., a human) a therapeutically effective amount of any of the compositions or viral vectors described herein. Also provided are methods of expressing a peptide that inhibits α-synuclein aggregation (e.g., any of the α-synuclein aggregation inhibiting peptides described herein) in a cell (e.g., a neuronal or neuroglial cell) in a mammal (e.g., a human) that include introducing into the central nervous system of the mammal a therapeutically effective amount of any of the viral vectors described herein. In some embodiments of methods of introducing into a cell any of the compositions or viral vectors described herein (e.g., a composition or viral vector that encodes a peptide that inhibits α-synuclein aggregation), expression of the peptide that inhibits α-synuclein aggregation is increased in the cell, e.g., as compared to expression of the peptide that inhibits α-synuclein aggregation in a reference cell (e.g., a reference cell into which the composition or viral vector has not been introduced). In some embodiments, the reference cell does not express the peptide that inhibits α-synuclein aggregation, or does not express a detectable amount of the peptide that inhibits α-synuclein aggregation.

Also provided are methods of treating a neurodegenerative disease in a subject (e.g., a human) that include administering a therapeutically effective amount of any of the compositions or viral vectors described herein (e.g., AAV vectors, e.g., an AAV vector that encodes a peptide that inhibits α-synuclein aggregation, e.g., any of the α-synuclein aggregation described herein) into the central nervous system of a subject. Also provided herein are methods for reducing α-synuclein aggregation in a subject in need thereof, the methods comprising administering to the subject an effective amount of any of the compositions or viral vectors (e.g., AAV vectors, e.g., an AAV vector that encodes a peptide that inhibits α-synuclein aggregation, e.g., any of the α-synuclein aggregation described herein) described herein.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease (e.g., Alzheimer's disease or a synucleinopathy). In some embodiments, “treatment” includes resolution of a particular disorder, including a reduction in one or more symptoms of the disorder and/or a reduction in in the severity of one or more symptoms associated with the disorder (e.g., Alzheimer's disease or a synucleinopathy).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human.

An “effective amount” or “therapeutically effective amount” of a therapeutic agent is any amount of the agent that, when used alone or in combination with one or more additional therapies, slows down the onset of a disorder (e.g., Alzheimer's disease or a synucleinopathy) or promotes regression of the disorder evidenced by a decrease in severity of disorder symptoms, an increase in frequency and duration of disorder symptom-free periods, or a ameliorating an impairment or disability due to the disorder affliction. The ability of one or more additional therapies to promote disorder regression can be evaluated using a variety of methods known to the skilled practitioner, such as in human subjects during clinical trials, in animal model systems predictive of efficacy in humans, or by assaying the activity of the agent in in vitro assays. In some embodiments, an effect amount of any of the viral vectors or compositions comprising viral vectors described herein is sufficient to cover a region of interest (e.g. substantia nigra or putamen) in a subject when the viral vector or composition is administered to the subject. In some embodiments, an effective amount of any of the viral vectors or compositions comprising viral vectors described herein is measured in genomic particles per brain (gp/brain, e.g., gp/human brain). In some embodiments, an effective amount of any of the viral vectors or compositions comprising viral vectors described herein is about 1×10⁹, about 2×10⁹, about 3×10⁹, about 4×10⁹, about 5×10⁹, about 6×10⁹, about 7×10⁹, about 8×10⁹, about 9×10⁹, about 1.0×10¹⁰, about 1.1×10¹⁰, about 1.2×10¹⁰, about 1.3×10¹⁰, about 1.4×10¹⁰, about 1.5×10¹⁰, about 1.6×10¹⁰, about 1.7×10¹⁰, about 1.8×10¹⁰, about 1.9×10¹⁰, about 2.0×10¹⁰, or about 3.0×10¹⁰ gp/brain.

“Administering” or “administration” refer to the physical introduction of a therapeutic agent to a subject, using any of the various methods and delivery systems described herein or otherwise known to those skilled in the art. Routes of administration can include, without limitation, intracranial, oral, intravenous, intranasal, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods. In some embodiments, a therapeutically effective amount of any of the compositions or viral vectors described herein is administered to a subject only once. In some embodiments in which a therapeutically effective amount of any of the compositions or viral vectors described herein is administered to a subject only once, cells transfected with the viral vector continue to produce a peptide encoded by the viral vector (e.g., a peptide that inhibits α-synuclein aggregation, e.g., any of the α-synuclein aggregation inhibiting peptides described herein) in all of the non-dividing cells the particles infect for the life of that cell (e.g., neuronal or neuroglial cells).

In some embodiments, the neurodegenerative disease is Alzheimer's disease or a synucleinopathy. Non-limiting examples of a synucleinopathy include a Lewy body disorder, Parkinson's disease (PD), PD with dementia (PDD), pure autonomic failure (PAF), and multiple system atrophy (MSA).

Any of the variety of methods described herein or otherwise known in the art for introducing any of the compositions described herein into a mammalian cell can be used (e.g., through the use of a viral vector, e.g., any of the viral vectors described herein).

The administration of the compositions or viral vectors described herein can be carried out in any convenient manner, including, without limitation, by aerosol inhalation, injection, ingestion, transfusion, implantation or transplantation. The compositions described herein can be administered to a subject intracranially, transarterially, subcutaneously, intradermally, intranodally, intramedullary, intramuscularly, by intravenous (i.v.) injection, or intraperitoneally. In one aspect, the compositions or viral vectors described herein are administered to a patient by intracranial injection.

Methods of detecting expression and/or activity of a peptide that inhibits α-synuclein aggregation are known in the art. In some embodiments, the level of expression of a peptide that inhibits α-synuclein aggregation can be detected directly (e.g., detecting the peptide or detecting the mRNA that encodes the peptide). Non-limiting examples of techniques that can be used to detect expression and/or activity of a peptide that inhibits α-synuclein aggregation directly include: real-time PCR, Western blotting, immunoprecipitation, immunohistochemistry, or immunofluorescence. In some embodiments, expression of a peptide that inhibits α-synuclein aggregation can be detected indirectly e.g., via tests that monitor a neurodegenerative disease such as PD. Non-limiting examples of tests that monitor PD are those tests that monitor bradykinesia, tremor, rigidity, and/or postural instability. In some embodiments, a subject that has been administered an effective amount of any of the compositions or viral vectors (e.g., AAV vectors, e.g., an AAV vector that encodes a peptide that inhibits α-synuclein aggregation) described herein exhibits reduced PD or neurodegenerative symptoms (e.g., in one or more of bradykinesia, tremor, rigidity, and/or postural instability) as compared to a subject that has not been administered an effective amount of any of the compositions or viral vectors described herein. In some embodiments, a subject that has been administered an effective amount of any of the compositions or viral vectors described herein exhibits one or more symptoms that are reduced by about 10%, about 20%, about 30%, about 40%, about 50%, about 60%, about 70%, about 80%, about 90%, about 95%, or more as compared to a subject that has not been administered an effective amount of any of the compositions or viral vectors described herein. In some embodiments, a subject that has been administered an effective amount of any of the compositions or viral vectors described herein exhibits no PD or neurodegenerative symptoms as compared to a subject that has not been administered an effective amount of any of the compositions or viral vectors described herein. In some embodiments, a subject that has been administered an effective amount of any of the compositions or viral vectors (e.g., AAV vectors, e.g., an AAV vector that encodes a peptide that inhibits α-synuclein aggregation) described herein exhibits reduced PD or neurodegenerative symptom(s) (e.g., in one or more of bradykinesia, tremor, rigidity, and/or postural instability) as compared to the symptom(s) exhibited by the subject prior to administration of the composition or viral vector. In some embodiments, a subject that has been administered an effective amount of any of the compositions or viral vectors described herein exhibits one or more symptom(s) that are reduced by about 10%, about 20%, about 30%, about 40%, about 50%, about 60%, about 70%, about 80%, about 90%, about 95%, or more as compared to the symptom(s) exhibited by the subject prior to administration of the composition or viral vector. In some embodiments, the subject that has been administered an effective amount of any of the compositions or viral vectors described herein exhibits no PD or neurodegenerative symptom(s) as compared to the symptom(s) exhibited by the subject prior to administration of the composition or viral vector.

### EXAMPLES

The disclosure is further described in the following examples, which do not limit the scope of the disclosure described in the claims.

### Example 1. AAV Vector Preparation and Packaging

### Transformation of E. coli Cells and Preparation of Plasmid DNA

SUBCLONING EFFICIENCY™ DH5α™ Competent Cells were used for the transformation. The PURELINK™ HiPure Plasmid Maxiprep Kit was used for plasmid preparation.

DH5α™ cells were thawed on ice. The cells were gently mixed with a pipette tip. 50 μL of cells for each transformation were aliquoted into a separate, chilled 1.5 mL microcentrifuge tube. 1 to 5 μL of plasmid were added to the cells and mixed gently. The tubes were incubated on ice for 30 minutes. The cells were heat-shocked by placing the tubes in a 37° C. water bath for 5 minutes. The tubes were then placed on ice for 2 minutes. 250 μL of pre-warmed Luria Broth (LB) medium was added, and the tubes were incubated at 37° C. for 30 minutes without agitation. 20 μL to 200 μL from each transformation were spread onto pre-warmed LB-ampicillin agar plates. The plates were incubated overnight in a 37° C. bacterial incubator.

One bacterial colony was selected and used to aseptically inoculate 90 mL of LB supplemented with 100 μg/mL ampicillin in a conical flask. The flask was placed in a bacterial shaker and incubated overnight at 37° C. with agitation (shaker set at 220 rpm).

The following day, the bacterial culture was transferred into two 50 mL tubes. The cells were pelleted by centrifugation at 8,000×g for 5 min at 4° C. The supernatant was carefully decanted from the cell pellet and discarded. The tubes were inverted on paper towels for a few minutes to remove residual supernatant. 5 mL of Resuspension buffer (R3) with RNase A were added to each tube, and the pellet was resuspended. The cells suspensions from the two tubes were combined.

10 mL of Lysis Buffer (L7) were added to the tube and the solution mixed by gently inverting the capped tube several times. The tube was incubated at room temperature for 5 minutes. 10 mL Precipitation Buffer (N3) were added and the solution mixed gently by inverting the capped tube several times until the mixture appears to be homogenous.

The tubes were centrifuged at 8,000×g for 30 minutes at 4° C. The supernatant was carefully transferred to a clean 50 mL tube while avoiding transfer of any precipitate. A HiPure maxi column was pre-equilibrated by applying 30 mL Equilibration buffer (EQI). The solution was allowed to drain by gravity flow.

The supernatant was loaded onto the equilibrated column. The solution in the column was allowed to pass through by gravity flow. The column was washed twice with 30 mL Wash Buffer (W8). The solution was allowed to drain by gravity flow. The flow through was discarded. To elute the plasmid from the column, a sterile 50 mL tube was placed under the column, 15 mL Elution Buffer (E4) were added to the column, and the solution was allowed to drain by gravity flow. 10.5 mL isopropanol was added to the eluate. The eluate was mixed well and centrifuged at 8,000×g for 30 minutes at 18° C. The supernatant was carefully removed and discarded. The DNA pellet was resuspended in 0.4 mL TE buffer. The DNA was transferred into a 1.5 mL microcentrifuge tube. 20 μL of 3 M sodium acetate and 1 mL 100% ethanol were added to the tube. The solution was mixed well and centrifuged at 17,000×g for 3 min at room temperature.

The supernatant was removed and discarded. The DNA pellet was washed in 0.7 mL 79% ethanol. The tube was centrifuged at 17,000×g for 1 min at room temperature. The supernatant was removed and the pellet was air-dried for 10 minutes. The purified DNA was then resuspended in in 200-500 μL TE buffer. The plasmid was stored at −20 μC.

### HEK293 Cell Culture

Complete DMEM was warmed to 37° C. Frozen HEK293 cells were removed from liquid nitrogen storage and immediately placed the tube into a 37° C. water bath. The cells were thawed for 30-60 seconds in the 37° C. water bath. The tube was transferred into a Class II Biological Safety Cabinet and the cell suspension was added to 10 mL of complete DMEM in a sterile 50 mL conical centrifuge tube.

The cryovial that had contained the cells was rinsed with 1 mL of complete DMEM and added to the cell suspension. The cell suspension was centrifuged at 300×g, at 20° C. for 5 min. The supernatant as discarded, and the pelleted cells were resuspended in 5 mL of complete DMEM with gentle pipetting. The cell suspension was transferred into a T175 flask containing 35 mL of complete DMEM The cells were grown and maintained in an incubator at 37° C. in 5% CO₂.

The HEK293 cells were split every 3-4 days when they grew to approximately 90% confluency. Cells may be passaged 20 times before a new batch of cells is required. Complete DMEM, 1×PBS solution, and TrypLE Express were warmed to 37° C. The culture medium was removed from the flask and discarded. 20 mL of 1×PBS was added to the flask and the flask was gently tilt back and forth to wash the cells. The PBS solution was removed and discarded.

5 mL of TrypLE Express were added to the cells. The cells were incubated at 37° C. for 5-10 minutes. The flask was gently swirled to detach the cell. 5 mL of complete DMPEM was added to the flask. The cell suspension was transferred from the flask to a sterile 50 mL conical centrifuge tube. The tube was centrifuged at 300×g at 20° C. for 5 minutes.

The supernatant was discarded, and the cell pellet was loosened by gently tapping the side of the tube. The cells were resuspended in 8 mL (for a 3-day cycle) or 10 mL (for a 4-day cycle) of complete DMEM and mixed gently. 1 mL of the cell suspension was transferred into a new T175 flask containing 35 mL of complete DMEM, i.e., a 1.8 subcultivation ratio for 3-day culture period or 1:10 subcultivation ratio for 4-day culture period. The cells were returned to an incubator at 37° C. in 5% CO₂.

### Cell Plating

On Day 1, HEK293 cells (human embryonic kidney cells 293) were seeded into 5×15 cm tissue culture dishes that were sufficient for a single 300 μL batch of rAAV. The number of cells in one 90% confluent T175 flask was sufficient to seed 3.5-4.0×15 cm plates (plating density of approximately 15×10⁶-20×10⁶ cells per plate) such that a confluency of 70% was achieved at the time of transfection. The cells were dissociated and harvested from the T175 flasks as described above.

The harvested cell pellet was resuspended in an appropriate volume of complete Dulbecco's Modified Eagle Medium (DMEM) to generate a cell suspension sufficient to generate 1×15 cm dish/mL (i.e., if one T175 flask generated 3.5×15 cm dishes, 3.5 mL of complete DMEM per T175 flask was used). 1 mL of cell suspension was added to 20 mL of complete DMEM in each 15 cm tissue culture dish and mixed thoroughly. The plates were returned to an incubator at 37° C. in 5% CO₂ making sure the plates were level.

### Transfection

The cellular transfection steps were conducted on Day 2. The plates were at 70% confluency at the time of transfection. Three hours prior to transfection the media from each plate was aspirated, discarded, and replaced with 20 mL of IMDM (Iscove's Modified Dulbecco's Medium).

Three hours prior to transfection the transfection reagents were brought to room temperature by removing aliquots of 2.5 M CaCl₂, HEBS (HEPES-buffered saline), and the plasmids (see above) from −20° C.

Immediately prior to transfection, the DNA mix was prepared by pipetting the reagents in the following order into a 50 mL tube: 12 mL sterile room temperature ultrapure water, 1.65 mL 2.5M CaCl₂, 62.5 μg pAAV expression plasmid, 125 μg pFΔ6, and 62.5 μg capsid plasmid.

The DNA mix was filter sterilized into a 75 cm² tissue culture flask using a 0.2 μm ACRODISC® syringe filter. For the transfection: while vigorously vortexing the DNA mix (in the flask), an optimized volume (6 to 13 mL) of HEBS was pipetted into the flask and vortexing was continued for 10-15 seconds.

The transfection mix was left to stand at room temperature for a further 2 min. The transfection mixture had a slightly opaque appearance due to the formation of a fine precipitate.

The 5×15 cm plates of HEK293 cells were removed from the incubator and 1/5^{th} of the transfection mix was gently pipetted into each plate in a circular drop-wise motion. This process was done carefully but expeditiously such that the mix was added to all plates within 5 min. The plates were gently agitated to ensure even distribution of transfection mix and returned to the tissue culture incubator. Approximately 16-18 hours later, the media was removed and discarded. 20 mL of pre-warmed (37° C.) complete DMEM was added to the plates and plates were returned to the incubator.

### Cell Harvesting and Sample Preparation

The cells were harvested about 65 hours after transfection. The media was aspirated and discarded. The cells were gently washed by pipetting about 20 mL pre-warmed (37° C.) 1× PBS (phosphate-buffered saline) onto each plate, the plate was gently swirled, and the PBS was removed and discarded.

Another 20 mL 1× PBS was added to each plate, and the cells were detached from the plate using a cell scraper. The cell suspension was collected in sterile 50 mL tubes (2 μlates per 50 mL tube and the suspension from the remaining plate in another 50 mL tube).

The tubes were centrifuged at 600×g at 4° C. for 30 minutes in a centrifuge equipped with a swing-out rotor. The supernatant was carefully removed and discarded. The cells were pooled by resuspending the cell pellets in lysis buffer to a final volume of 8 mL per batch of five plates.

10% sodium deoxycholate was added to a final concentration of about 0.5% (400 μL of 10% sodium deoxycholate in 8 mL) and BENZONASE® to a final concentration of 50 U/mL. The solution in the tubes was mixed well, the tubes were placed in a 37° C. water bath for 60 min. The tubes were vortexed every 15 minutes during the incubation period. The lysates were stored at −20° C.

### Iodixanol Gradient Preparation

The crude rAAV vector-containing cell lysates were thawed to room temperature. The lysate was centrifuged at 7,000×g at 4° C. for 15 min to remove any particulate matter. The cell supernatant was transferred into a fresh tube.

The iodixanol gradient was prepared in a disposable 35 mL ultracentrifuge tube. The solutions were loaded in the following order suing a spinal needle attached to a 10 mL disposable syringe: 7.5-8.0 mL of cell supernatant; 8.5 mL 15% iodixanol; 6.0 mL 25% iodixanol; 5.0 mL 40% iodixanol; and 5.0 mL 54% iodixanol. The needle was held against the side of the tube and each solution was slowly expelled so as to not disturb the layer above. Lysis buffer (150 mM NaCl, 50 mM Tris, pH 8.5) was added to the cell supernatant at the top of tube to within 1-2 mm below the bottom of the tube stopper, being careful not to disturb any of the layers.

The tube was centrifuged in a SORVALL™ ultracentrifuge T865 rotor at 58,000 rpm for 1 hour and 45 minutes (including the 15 minute acceleration and 90 minute spin). An 18 gauge needle attached to a 5 mL syringe was inserted at the 40-5 4% iodixanol interface. A 20 gauge needle was inserted at the top of the tube to facilitate entry of air into the tube. With the bevel of the 18 gauge needle facing upwards, 3.5-4.0 mL of the 40% iodixanol layer containing the vector was carefully drawn off and transferred to a 50 mL tube.

10 mL of 0.001% pluronic acid in 1× PBS—MgCl₂ was added to the vector sample before filtering the sample through a 0.45 μm syringe filter into a 15 mL 100 kDa molecular weight cut-off AMICON® concentrator. The sample was centrifuged at 3,000×g until the sample volume was reduced to about 200 μL.

14-15 mL of 0.001% pluronic acid in 1× PBS-MgCl₂ was added to the concentrated and centrifuged as above. This was repeated 3-4 times until the iodixanol was removed. On the final wash, the sample was centrifuged to a volume of about 200 μL and pipetted into a 1.5 mL tube. 150 μL of 0.001% pluronic acid in 1× PBS-MK was added to the concentrator to rinse the inside, which was combined with the sample to give a total of about 350 μLAAV vector.

The sample was filter sterilized through a 13 mm, 0.2 μm, low protein biding ACRODISC® filter with a HT TUFFRYN® Membrane into a new sterile 1.5 mL protein LOBIND® tube. The vector was stored at −80° C. for long term storage. SDS-PAGE

To confirm the presence of the three rAAV capsid proteins (VPI, 2, and 3) and to check for the presence/absence of other protein contaminants, the rAAV samples were analyzed by SDS-PAGE using the mini-PROTEAN 3 system (Bio-Rad) for gel preparation and electrophoresis.

rAAV Vector Titration

rAAV vector preparations can contain a mixture of empty capsids and genome-containing vector particles. To standardize the amount of DNA-containing vector particles used, the genomic titers of viral vector stocks can be determined by SYBR Green-based qPCR using the standard curve method. Vector DNA extracted from the intact rAAV particles was measured against a standard curve generated using a reference plasmid of known molarity that has been serially diluted over a range of 10³ to 10⁷ μlasmid copies per μL. An NTC of PCR grade water was also included and each reaction was performed in triplicate. A sample of rAAV vector stock of known titer (titering control or reference standard) can also be included in each run to allow standardization of titers between runs. However, if direct comparisons are to be made between vectors, titers should be determined in the same qPCR experiment

Sufficient DNase I master mix was prepared for triplicate DNA extractions for each unknown rAAV sample and rAAV reference standard (if included) and one NTC reaction. The reactions were set up in 0.2 mL PCR tubes by combining 48 μL of DNase I master mix and 2 μL of unknown rAAV or rAAV reference standard. 2 μL of ultrapure water or PCR grade water were added to the NTC reaction instead of rAAV The contents of the tubes was mixed, and the tubes were briefly spun.

The tubes were incubated for 15 min at 25° C. to remove any contaminating DNA not packaged within rAAV virions. All incubation steps were carried out in a thermocycler with a 0.2 mL block. 1 μL of 50 mM EDTA was added to each tube, the contents of the tubes were mixed, and the tubes were briefly spun. The DNase I was heat-inactivated at 70° C. for 10 min. The viral capsids were digested by addition of 49 μL of proteinase K master mix to each 51 μL DNase I digest. The samples were briefly vortexed and centrifuged. The samples were then incubated at 55° C. for 1 hour. The proteinase K was inactivated by heating at 95° C. for 20 minutes. The samples were stored at 4° C. until required for qPCR.

To prepare plasmid standards, a dilution series of 1 ×10³ to 1 ×10⁷ copies/μL of a plasmid of known size containing the amplifiable sequence was generated. The plasmid was diluted to 25-100 ng/μL using sterile TE buffer or PCR grade water and the concentration was determined using a high accuracy spectrophotometer. The mass of the plasmid was also determined as well as the concentration in copies/μL of the plasmid stock. The plasmid was diluted in TE buffer or PCR-grade water to generate a 1 ×10⁹ copies/μL stock. A 10-fold serial dilution of the stock in TE buffer or PCR-grade buffer was also made to generate plasmid standards of 1 ×10³, 1 ×10⁴, 1 ×10⁵, 1 ×10⁶ and 1×10⁷ copies/μL. These were stored as 100 μL aliquots at −80° C. A new set of standards was thawed for each qPCR run.

For qPCR, 100 μL of ultrapure or PCR-grade water were added to the 100 μL of rAAV DNA samples from above. The 200 μL samples were vortexed and briefly spun. A 1:5 to 1:50 dilution of each triplicate vector DNA preparation from above was generated. The samples were vortexed and briefly spun.

Sufficient SY3R Green PCR master mix for triplicate PCR reactions of each plasmid standard, vector DNA sample, and NTC were prepared. Three replicate 125 μL PCR reactions were set up for each rAAV DNA sample, plasmid standard and NTC that comprised 10 μL of Green PCR master mix and 2.5 μL of sample in wells of a 384-well optical reaction plate. The plate was sealed with an optical adhesive cover and protect from light by wrapping in aluminum foil. The plate was stored at 4° C. until the end of run.

The plate was centrifuged for 2 min at 20° C. at 1700× g and transferred to a real-time thermal cycler (Applied Biosystem 7900HT real-time PCR system) and real-time PCR was run using the following cycling conditions:

| | | | | |
|---|---|---|---|---|
| Real time amplification | Step 1 (1 cycle) | 2 | min | 50° C. |
| | Step 2 (1 cycle) | 10 | min | 95° C. |
| | Step 3 (40 cycles) | 15 | sec | 95° C. |
| | | 1 | min | 60° C. |
| Melting Curve Analysis | Step 4 (1 cycle) | 15 | sec | 95° C. |
| | | 15 | sec | 60° C. |
| | | 15 | sec | 95° C. |

The data were analyzed using the instrument software. Adjustment of the baseline and threshold settings were manually adjusted as needed. The amplification curves of each set of three replicates were checked and any wells that showed amplification variation were omitted. The standard curve of Ct versus log input copies of standard plasmid were evaluated. The slope of the standard curve was close to 3.2 and the correlation coefficient (R² value) was 0.99 otherwise the run was repeated with a new set of standards.

The mean quantity (starting copies per reaction) for triplicate DNA preparations of unknown vector sample were determined, and the genomic titer (vector genomes (vg)/mL) of the original vector was calculated.

### Example 2. Study of the Neuroprotective Potential of AAV1/2 Encoded Peptides in the Pre-Formed α-Synuclein Fibril Rat Model of Parkinson's Disease

### Study Overview

This is a non-GLP study to assess the ability of S62 and S71, delivered via AAV1/2 viral vector, to protect dopaminergic function in the pre-formed α-synuclein (aSyn) fibril rat model of PD.

Production of AAV1/2 vectors encoding S62 and S71 will be completed.

On day—14 (D-14), animals will receive a stereotaxic injection of AAV1/2-EV (Groups 1 and 2), AAV1/2-S62 24MER (SEQ ID NO: 6, Group 3) or AAV1/2-S71 22MER (SEQ ID NO: 7, Group 4) into the right substantia nigra (SN). Injection volume will be 2 μl per site at a final vector titer to be decided.

On D1, animals will receive a stereotaxic injection of either mouse sequence monomer control (mMC, Group 1, StressMarq, BC, Canada, 8 μg/ml) or mouse sequence aSyn PFFs (mPFF Type-1, StressMarq, 8 μg/ml) into the right striatum at two sites (2×2 μl of 8 μg/μl mPFF=32 μg total).

The Study will incorporate a total of four treatment groups with N=12 animals per group (total N=48, female Sprague-Dawley rats, Charles River, Canada).
Group 1 AAV1/2-scr+mMC N=12
Group 2 AAV1/2-scr+mPFF N=12
Group 3 AAV1/2-S62+mPFF N=12
Group 4 AAV1/2-S71+mPFF N=12

On D120, animals will be killed and brain samples will be collected, processed and stored for further analysis.

Primary post mortem measures will include:
Qualitative assessment of pSer129 aSyn expression for extent of pathological aSyn spread in the forebrain, including cortical regions, by IHC.
Quantitative assessment of tyrosine hydroxylase positive neurons and/or pSer129-aSynpositive neurons in the SN by IHC and stereology.
Qualitative assessment of expression and distribution of the novel peptides (HA immunoreactivity) within the dopaminergic (TH immunoreactivity) nigrostriatal pathway by IF.

Secondary post mortem measures may include,
Quantitative assessment, by way of stereology, of pSer129 aSyn positive cortica neurons within a defined region of the cortex (e.g. parietal cortex, cingulate cortex insular cortex) supported by the qualitative assessment of pSer129 aSyn expression in Group 2 (AAV1/2-scr+mPFF).

### Animal Welfare

animal studies are conducted according to CCAC guidelines and under IACUC-approved Animal Use Protocols (AUPs).

Throughout the Study, an animal shall be euthanized immediately if any unforeseen adverse event affects an animal resulting in distress, injury, weight loss exceeding 20% of starting weight or any of the following humane endpoints.
abnormal posture (hunched)
failure to groom
lethargic (reduced activity or responsiveness)
impaired ambulation or paralysis
abnormal vocalization
persistent anorexia and dehydration that cannot be alleviated
hemorrhagic discharge
abdominal distention causing distress/pain
dyspnea
cardiovascular collapse
persistent self-trauma
tumor size exceeding 1.5 cm in adult mice
ulcerated tumors
pre-moribund
moribund (severe depression, unable to respond to external stimuli, non-ambulatory, inability to right itself)

When euthanasia is required for a given animal, the animals will be euthanized according to the methods approved in the Animal Use Protocol (AUP) or in consultation with the Clinical Veterinarian. The methods chosen will be compliant with those approved by the Institutional Animal Care and Use Committee (IACUC) and will be species specific. The recommendations for euthanasia are, in large part, based upon the 2013 AVMA guidelines on euthanasia and CCAC Guidelines on the Euthanasia of Animals Used in Science (2010).

In this study, depending on circumstances, such as the availability of staff and the urgency of euthanasia, the chosen method of euthanasia will be induction of general anesthesia using isoflurane followed by cervical decapitation, or perfusion under general anesthesia, using isoflurane.

### Vehicle and Animal Husbandry

Production of AAV1/2 vectors encoding each of the two novel peptides, S62 and S71, incorporating hemagglutinin (HA) tags, will be completed. The vehicle for this study will be sterile PBS should dilution be required. Empty vector will be used as the control for each vector used.

The Study will use a total of 48 female Sprague-Dawley rats (Charles River, 275-300 g at time of first surgery). Animals will be housed 2 per cage and acclimatised for at least one week prior to start of any procedures. Animals will be housed at standard temperature (21±2° C.) in a light controlled environment (lights on 7.00 am to 7.00 pm) with access to food (Teklad 7912, Harlan, Madison, WI) and water ad libitum. Animals will be weighed on the first day of acclimatization and then on a weekly basis thereafter.

### Surgeries

Animals will receive a series of stereotaxic injections, first into the substantia nigra (D-14) and PFFs into the striatum on D1.

### Day—14 (AAV1/2-S62 or AAV1/2-S71)

On Study D-14, animals will receive a unilateral intranigral administration at a single site of either empty vector (AAV1/2-EV; Groups 1 and 2), AAV1/2-S62 (SEQ ID NO. 6, Group 3) or AAV1/2-S71 (SEQ ID NO. 7, Group 4) according to stereotaxic techniques. Injection volume will be 2 μl per site at a concentration of 3.0 ×10¹² gp/ml. All vectors will be diluted (if required) in PBS. The injection site will be −5.2 mm AP, and −2 mm ML relative to Bregma (according to the atlas of Paxinos and Watson, 1986) with the needle lowered −7.5 mm below the skull.

Day 1 (mPFF)

On D1, two weeks following administration of vectors encoding each of the two peptides, a second stereotaxic surgery will be conducted. Animals will receive either mouse sequence monomer control (mMC, Group 1, StressMarq, 8 g/ml) or mouse sequence aSyn PFFs (mPFF Type-1, StressMarq, 8 g/ml) administered unilaterally into the striatum at each of two-sites (2×2 1 of 8 g/1 mPFF=32 g total). The injection sites will be +1.6 mm AP and +2 mm ML with the needle lowered 4 mm from skull (site 1) and −0.1 mm AP, +4.2 mm ML with the needle lowered 5 mm from skull (site 2).

### Surgical Overview (all Surgeries)

All surgeries will be performed using aseptic technique. Under isoflurane anesthesia (2% with 2 L/min oxygen flow rate, isoflurane USP 99.9%) and after confirmation of loss of tail-pinch and corneal reflexes, rats will be placed in a Kopf small animal stereotaxic frame with the incisor bar set 3.3 mm below the ear bars (interaural line). The animal's head will be shaved and the skin cleaned thoroughly using disinfectant soap, isopropyl alcohol 70% USP and iodine surgical scrub (7.5% povidone iodine). An incision (˜2 cm) will be made with a sterile scalpel blade in an anteroposterior direction along the midline. After exposure of Bregma using a cotton-bud, burr holes will be drilled in the skull above either the SN (Day—14) or striatum (Day 1) at the coordinates described above according to the atlas of Paxinos and Watson (1986). A customized, 1 inch, 26G Hamilton needle with 45-degree bevel will be used for all injections. After each injection the needle will be left in place for an additional 5 min to ensure complete absorption of the solution. After slow retraction of the needle following the final injection, the incision will be closed by means of wound clips and animals will be administered saline (50 ml/kg, SC). The wound will be coated with analgesic, antibacterial cream. Finally, animals will be removed from the frame and placed in a recovery cage, positioned atop a thermostatically-controlled pad, and monitored until conscious.

### Treatment Groups

The study will incorporate 4 groups of rats. Animals will be assigned to each group randomly at the beginning of the Study. Treatments will be as shown in Table 2.

TABLE 2

| | AAV1/2 | mMC or | Terminal Procedures | |
|---|---|---|---|---|
| | Test Item | mPFF | D 120, kill, | |
| Group | D −14 i.c. | D 1 i.c. | brain dissection | N |
| 1 | AAV1/2-scr | mMC | ✓ | 12 |
| 2 | AAV1/2-scr | mPFF | ✓ | 12 |
| 3 | AAV1/2-S62 | mPFF | ✓ | 12 |
| 4 | AAV1/2-S71 | mPFF | ✓ | 12 |

AAV1/2-scr: AAV1/2-scrambled S62 with HA-tag, 3.0 × 10¹² gp/ml
AAV1/2-S62: AAV1/2-S62 24mer with HA-tag, 3.0 × 10¹² gp/ml
AAV1/2-S71: AAV1/2-S71 22mer with HA-tag, 3.0 × 10¹² gp/ml
mMC: monomer control
mPFF: mouse sequence aSyn pre-formed fibrils

### Necropsy and Tissue Preparation

On D120, animals will receive an overdose of isoflurane and be killed via exsanguination by way of transcardial perfusion with ice-cold 0.9% saline containing 0.2% heparin. Brains will be rapidly removed and then be immersed in 4% paraformaldehyde (PFA) for 48 hours for fixation followed by cryoprotection in graded sucrose solutions (15 to 30% sucrose). All tissue will be stored at −80° C. in a locked freezer.

pSer129 aSyn Immunohistochemistry

Post-fixed and cryo-preserved sections of forebrain and midbrain will be processed for pSer129 aSyn immunoreactivity. Brains will be cut frozen in the coronal plane at a thickness of 40 μm on a sledge microtome (Leica) and 6 series of sections will be stored in cryoprotectant. One series of sections will be processed for visualization of pSer129 aSyn via the biotin-labeled antibody procedure. Briefly, following several washes in a PBS solution containing 0.1% Tween-20, endogenous peroxidase will be quenched in a 3% hydrogen peroxide solution. Antigen retrieval will be performed by incubating the sections in a sodium citrate buffer for 1 hr at 37° C. followed by 3 rinses in PBS. Background staining inhibited in a 5% normal donkey serum/2% bovine serum albumin solution. Tissue will be then incubated overnight with anti pSer129 aSyn (1:5000; Abcam, ab51253) After three washes in PBS, sections will be sequentially incubated in biotinylated antibodies IgG (donkey anti-rabbit, 1:500; Jackson, West Grove, PA) and the Elite avidin-biotin complex (ABC Kits; Vector, Burlingame, CA) for 1 h separated by three washes in PBS. pSer129 aSyn immunostaining will be visualized following a reaction using 3,3-diaminobenzidine (ABC Elite, Vector Laboratories, Cat. #PK-6100). Sections will be then mounted on glass slides, allowed to dry, dipped into dH₂O, dehydrated through graded alcohols (70%, 95%, 100%) and cleared in histo-clear prior to being coverslipped with Vecta-mount mounting medium.

Expression of pSer129 aSyn will be qualitatively assessed in the forebrain for extent of pathological aSyn spread. In addition, a quantitative assessment of pSer129-aSyn positive neurons in the SN will be performed by stereology (see below). Based on the outcome of the qualitative assessment of the cortex, it may be determined that a quantitative assessment should follow. Thus, by way of stereology, pSer129 aSyn positive cortical neurons within a defined region of the cortex (e.g. parietal cortex, cingulate cortex, insular cortex) will be conducted. The region of interest will be supported and defined by the qualitative assessment of pSer129 aSyn expression in Group 2 (AAV1/2-scr+mPFF).

### Immunohistochemistry and Stereology for Nigral TH

Brains will be sectioned frozen in the coronal plane at a thickness of 40 μm on a freezing sliding microtome (Leica Microsystems Inc., Richmond Hill, ON) and 6 series of sections will be stored in cryoprotectant (30% glycerol, 30% ethoxyethanol, 40% PBS). A single series of sections will be processed for visualization of tyrosine hydroxylase (TH) via the biotin-labelled antibody procedure. Briefly, following several washes in a PBS solution containing 0.2% Triton X-100 (PBS-T), endogenous peroxidase will be quenched in a 3% hydrogen peroxide solution and background staining inhibited in a 10% normal goat serum/2% bovine serum albumin solution. Tissue will then be incubated with primary antibodies overnight: rabbit anti-TH antibody (1:1000, Pel-Freez, Rogers, AR). After three washes in PBS-T, sections will be sequentially incubated in biotinylated goat anti-rabbit or mouse IgG (1.300; Vector, Burlingame, CA) for 1 h and the Elite avidin-biotin complex (ABC Kits; Vector, Burlingame, CA) for 1 h separated by three washes in PBS. Immunostaining will then be visualized following a reaction with 3,3-diaminobenzidine (Vector, Burlingame, CA). Sections will be mounted on glass slides, allowed to dry, dipped into dH₂O, dehydrated through graded alcohols (70%, 95%, 100%), cleared in xylenes, and coverslipped with DPX mounting medium (Electron Microscopy Sciences, Hatfield, PA).

Tyrosine hydroxylase positive neurons will be quantitatively assessed in the SN by stereology.

Estimates of TH^{+ve} neuronal number and/or pSer129^{+ve} neuronal number within the substantia nigra pars compacta (SNc) will be performed using Stereo Investigator software (MBF Bioscience, Williston, VT) according to stereologic principles. Six to eight sections, each separated by 240 m from the anterior to the posterior SN, will be used for counting each case. Stereology will be performed using a Zeiss microscope (Carl Zeiss, Canada) coupled to a digital camera for visualisation of tissue sections. The total number of TH^{+ve} and/or pSer129-aSyn neurons will be estimated from coded slides using the optical fractionator method. For each tissue section analysed, section thickness will be assessed empirically and guard zones of ˜2 m thickness will be used at the top and bottom of each section. The SNc will be outlined under low magnification (5×) and neurons counted under 40× magnification. Stereological parameters will be empirically determined (i.e. grid size, counting frame size and dissector height) using Stereo Investigator software (MicroBrightfield, VT, USA). The acceptable coefficient of error (CE) will be calculated according to the procedure of West and colleagues, known as the Gunderson CE (m=1). Gunderson values <0.10 will be accepted.

### Tyrosine Hydroxylase+Hemagglutinin Immunofluorescence

Using a single series of midbrain and caudal striatal sections, double label immunofluorescence will be performed to reveal hemagglutinin (HA)-tagged human aSyn and tyrosine hydroxylase (TH). Briefly, on free floating sections, levels and distribution of TH (sheep anti-TH, 1:1000, Pel Freez, P60101; secondary antibody, Alexa fluor donkey anti-sheep, Fisher Scientific, A21099, 1:500) and HA (rabbit anti-HA, 1:1000; Abcam, AB9110; Alexa Fluor donkey anti-rabbit, 1:500, Fisher Scientific, A21206, 1:500) will be evaluated by double label immunofluorescence.

HA-tagged human aSyn and TH will be qualitatively assessed for extent of expression and distribution of the novel peptides (HA immunoreactivity) within the dopaminergic (TH immunoreactivity) nigrostriatal pathway.

### Statistical Analyses

Continuous data derived from post mortem endpoints (nigral TH+ve cell counts) will be graphed as mean±SEM and analysed using one-way or two-way analysis of variance (RM-ANOVA) followed by an appropriate post hoc Multiple Comparison Test using GraphPad Prism (v.8).

### Example 3. Stability and Permeability Studies of 562 and 571 Peptides

### Stability in Rat Plasma

S62 and S71 peptides (SEQ ID NOs: 3 and 4, respectively) were incubated in vitro in in rat plasma with 1% TCA and methanol as quenchers for a period of time calculated to be reflective of the amount of time the peptides would be subject to in vivo conditions if administered to rats. The recovery of the S62 and S71 peptides was not high enough to detect.

### Permeability in a Caco-2 Assay

Permeability of the S62 and S71 peptides were assessed in a Caco-2 assay using methanol. As can be seen in Table 3, neither of the peptides exhibited permeability.

TABLE 3

| | | Mean | Mean | Mean | Mean | Mean | |
|---|---|---|---|---|---|---|---|
| | | Papp | Papp | (B- | A- | B- | |
| | Assay | A-B | B-A | A/A-B) | B | A | A-B |
| Compound | Conc. | (10-6 | (10-6 | Efflux | % | % | Permeability |
| ID | (μM) | cm/s) | cm/s) | Ratio | Recovery | Recovery | Ranking |
| S62 | 10 | 0.00 | 0.00 | NA | 70.1% | 92.5% | Compound |
| | | | | | | | not |
| | | | | | | | detected in |
| | | | | | | | T120 Basal |
| | | | | | | | or T120 |
| | | | | | | | Apical side |
| S62 | 10 | 0.00 | 0.00 | NA | 59.7% | 91.3% | Compound |
| Verapamil | | | | | | | not |
| 25 | | | | | | | detected in |
| | | | | | | | T120 Basal |
| | | | | | | | or T120 |
| | | | | | | | Apical side |
| S71 | 10 | 0.00 | 0.00 | NA | 91.4% | 74.7% | Compound |
| | | | | | | | not |
| | | | | | | | detected in |
| | | | | | | | T120 Basal |
| | | | | | | | or T120 |
| | | | | | | | Apical side |
| S71 | 10 | 0.00 | 0.00 | NA | 80.7% | 92.7% | Compound |
| Verapamil | | | | | | | not |
| 25 | | | | | | | detected in |
| | | | | | | | T120 Basal |
| | | | | | | | or T120 |
| | | | | | | | Apical side |

Controls:

| | | | | | | | |
|---|---|---|---|---|---|---|---|
| Warfarin | 10 | 36.8 | 33.8 | 0.917 | 93.0% | 93.4% | Higher as |
| | | | | | | | expected |
| Talinolol | 10 | 0.404 | 6.09 | 15.1 | 86.3% | 101.7% | Effluxed as |
| | | | | | | | expected |
| Talinolol | 10 | 0.800 | 1.91 | 2.39 | 83.2% | 94.2% | Efflux |
| Verapamil | | | | | | | Inhibited by |
| 25 uM | | | | | | | Verapamil |
| | | | | | | | as |
| | | | | | | | expected |
| Ranitidine | 10 | 0.391 | 1.03 | 2.65 | 87.1% | 95.2% | Lower as |
| | | | | | | | expected |

Permeability Ranking: lower is <1×10−6 cm/s; higher is >1×10-6 cm/s. An efflux ratio >2 indicates potential for the compound to be a substrate for Pgp or other active transporter.

SEQUENCES

SEQ ID NO: 1
AVVWGVTAV
SEQ ID NO: 2
AVVTGVTAV
SEQ ID NO: 3
GAVVWGVTAVKK
SEQ ID NO: 4
RAVVTGVTAVAE
SEQ ID NO: 5
GAVVWGVTAVKKKKK
SEQ ID NO: 6
GAVVWGVTAVKKGRKKRRQRRRPQ
SEQ ID NO: 7
YGRKKRRQRRRAVVTGVTAVAE
SEQ ID NO: 8
MDVFMKGLSKAKEGVVAAAEKTKQGVAEAAGKTKEGVLYVGSKTKEGVVHGV
ATVAEKTKEQVTNVGGAVVTGVTAVAQKTVEGAGSIAAATGFVKKDQLGKNEE
GAPQEGILEDMPVDPDNEAYEMPSEEGYQDYEPEA
SEQ ID NO: 9
agcttgcatgcctgcaggtcactgttctcatcacatcatatcaaggttatataccatcaatattgccacagatgttacttagccttttaat
atttctctaatttagtgtatatgcaatgatagttctctgatttctgagattgagtttctcatgtgtaatgattatttagagtttctctttcatctg
ttcaaatttttgtctagttttattttttactgatttgtaagacttctttttataatctgcatattacaattctctttactggggtgttgcaaatat
tttctgtcattctatggcctgacttttcttaatggttttttaattttaaaaataagtcttaatattcatgcaatctaattaacaatcttttctttg
tggttaggactttgagtcataagaaatttttctctacactgaagtcatgatggcatgcttctatattattttctaaaagatttaaagttttgcctt
ctccatttagacttataattcactggaatttttttgtgtgtatggtatgacatatgggttcccttttattttttacatataaatatatttccctgt
ttttctaaaaaagaaaaagatcatcattttcccattgtaaaatgccatatttttttcataggtcacttacatatatcaatgggtctgtttctga
gctctactctattttatcagcctcactgtctatccccacacatctcatgctttgctctaaatcttgatatttagtggaacattctttcccattt
tgttctacaagaatatttttgttattgtctttgggctttctatatacattttgaaatgaggttgacaagttggggatcgtacctaatatcaca
aactggaaatgtctatcaatatatagttgctctagttattaatagtaatcaattacggggtcattagttcatagcccatatatggagttcc
gcgttacataacttacggtaaatggcccgcctggctgaccgcccaacgacccccgcccattgacgtcaataatgacgtatgttcc
catagtaacgccaatagggactttccattgacgtcaatgggtggactatttacggtaaactgcccacttggcagtacatcaagtgta
tcatatgccaagtacgccccctattgacgtcaatgacggtaaatggcccgcctggcattatgcccagtacatgaccttatgggact
ttcctacttggcagtacatctacgtattagtcatcgctattaccatggtcgaggtgagccccacgttctgcttcactctccccatctcc
cccccctccccacccccaattttgtatttatttattttttaattattttgtgcagcgatgggggcggggggggggggggggcgcgcg
ccaggcggggcggggcggggcgaggggcggggcggggcgaggcggagaggtgcggcggcagccaatcagagcggcg
cgctccgaaagtttccttttatggcgaggcggcggcggcggcggccctataaaaagcgaagcgcgcggcgggcgggagtcg
ctgcgacgctgccttcgccccgtgccccgctccgccgccgcctcgcgccgcccgccccggctctgactgaccgcgttactccc
acaggtgagcggggggacggcccttctcctccgggctgtaattagcgcttggtttaatgacggcttgtttcttttctgtggctgcg
tgaaagccttgaggggctccgggagggccctttgtgcggggggagcggctcggggctgtccgcggggggacggctgccttc
gggggggacggggcagggcggggttcggcttctggcgtgtgaccggcggctctagagcctctgctaaccatgttcatgccttc
ttctttttcctacagctcctgggcaacgtgctggttattgtgctgtctcatcattttg
SEQ ID NO: 10
tcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggcccgt
tgtcaggcaacgtggcgtggtgtgcactgtgtttgctgacgcaacccccactggttggggcattgccaccacctgtcagctccttt
ccgggactttcgctttccccctccctattgccacggcggaactcatcgccgcctgccttgcccgctgctggacaggggctcggct
gttgggcactgacaattccgtggtgttgtcggggaaatcatcgtcctttccttggctgctcgcctgtgttgccacctggattctgcgc
gggacgtccttctgctacgtcccttcggccctcaatccagcggaccttccttcccgcggcctgctgccggctctgcggcctcttcc
gcgtcttcgccttcgccctcagacgagtcggatctccctttgggccgcctccccgc
SEQ ID NO: 11
aataaa
SEQ ID NO: 12
agcttgcatgcctgcaggtcactgttctcatcacatcatatcaaggttatataccatcaatattgccacagatgttacttagccttttaat
atttctctaatttagtgtatatgcaatgatagttctctgatttctgagattgagtttctcatgtgtaatgattatttagagtttctctttcatctg
ttcaaatttttgtctagttttattttttactgatttgtaagacttctttttataatctgcatattacaattctctttactggggtgttgcaaatat
tttctgtcattctatggcctgacttttcttaatggttttttaattttaaaaataagtcttaatattcatgcaatctaattaacaatcttttctttg
tggttaggactttgagtcataagaaatttttctctacactgaagtcatgatggcatgcttctatattattttctaaaagatttaaagttttgcctt
ctccatttagacttataattcactggaatttttttgtgtgtatggtatgacatatgggttcccttttattttttacatataaatatatttccctgt
ttttctaaaaaagaaaaagatcatcattttcccattgtaaaatgccatatttttttcataggtcacttacatatatcaatgggtctgtttctga
gctctactctattttatcagcctcactgtctatccccacacatctcatgctttgctctaaatcttgatatttagtggaacattctttcccattt
tgttctacaagaatatttttgttattgtctttgggctttctatatacattttgaaatgaggttgacaagttggggatc
SEQ ID NO: 13
tagctgcgcgctcgctcgctcactgaggccgcccgggcaaagcccgggcgtcgggcgacctttggtcgcccggcctcagtga
gcgagcgagcgcgcagagagggagtggccaactccatcactaggggttccttgtagttaatgattaacccgccatgctacttatc
tacgtagccatgctctaggtacgatccccagcttgcatgcctgcaggtcactgttctcatcacatcatatcaaggttatataccatca
atattgccacagatgttacttagccttttaatatttctctaatttagtgtatatgcaatgatagttctctgatttctgagattgagtttctcat
gtgtaatgattatttagagtttctctttcatctgttcaaatttttgtctagttttattttttactgatttgtaagacttctttttataatctgcat
attacaattctctttactggggtgttgcaaatattttctgtcattctatggcctgacttttcttaatggttttttaattttaaaaataagtcttaa
tattcatgcaatctaattaacaatcttttctttgtggttaggactttgagtcataagaaatttttctctacactgaagtcatgatggcatgcttct
atattattttctaaaagatttaaagttttgccttctccatttagacttataattcactggaatttttttgtgtgtatggtatgacatatgggttc
ccttttattttttacatataaatatatttccctgtttttctaaaaaagaaaaagatcatcattttcccattgtaaaatgccatatttttttcatag
gtcacttacatatatcaatgggtctgtttctgagctctactctattttatcagcctcactgtctatccccacacatctcatgctttgctcta
aatcttgatatttagtggaacattctttcccattttgttctacaagaatatttttgttattgtctttgggctttctatatacattttgaaatgagg
ttgacaagttggggatcgtacctaatatcacaaactggaaatgtctatcaatatatagttgctctagttattaatagtaatcaattacgg
ggtcattagttcatagcccatatatggagttccgcgttacataacttacggtaaatggcccgcctggctgaccgcccaacgacccc
cgcccattgacgtcaataatgacgtatgttcccatagtaacgccaatagggactttccattgacgtcaatgggtggactatttacgg
taaactgcccacttggcagtacatcaagtgtatcatatgccaagtacgccccctattgacgtcaatgacggtaaatggcccgcctg
gcattatgcccagtacatgaccttatgggactttcctacttggcagtacatctacgtattagtcatcgctattaccatggtcgaggtga
gccccacgttctgcttcactctccccatctcccccccctccccacccccaattttgtatttatttattttttaattattttgtgcagcgatg
ggggcggggggggggggggggcgcgcgccaggcggggggggcggggcgaggggcggggggggcgaggcggag
aggtgcggcggcagccaatcagagcggcgcgctccgaaagtttccttttatggcgaggcggcggcggcggcggccctataaa
aagcgaagcgcgcggggggggagtcgctgcgacgctgccttcgccccgtgccccgctccgccgccgcctcgcgccgcc
cgccccggctctgactgaccgcgttactcccacaggtgagcggggggacggcccttctcctccgggctgtaattagcgcttgg
tttaatgacggcttgtttcttttctgtggctgcgtgaaagccttgaggggctccgggagggccctttgtgcggggggagcggctcg
gggctgtccgcggggggacggctgccttcgggggggacggggcagggcggggttcggcttctggcgtgtgaccggcggct
ctagagcctctgctaaccatgttcatgccttcttctttttcctacagctcctgggcaacgtgctggttattgtgctgtctcatcattttgg
caaagaattggatccactcgagttagccatgggcgccgtggtgtggggcgtgaccgccgtgaagaagggcaggaagaagag
gaggcagaggaggaggccccagagatcttatccgtatgatgttcctgattatgcttgatagtaagcttatcgataatcaacctctgg
attacaaaatttgtgaaagattgactggtattcttaactatgttgctccttttacgctatgtggatacgctgctttaatgcctttgtatcatg
ctattgcttcccgtatggctttcattttctcctccttgtataaatcctggttgctgtctctttatgaggagttgtggcccgttgtcaggcaa
cgtggcgtggtgtgcactgtgtttgctgacgcaacccccactggttggggcattgccaccacctgtcagctcctttccgggacttt
cgctttccccctccctattgccacggcggaactcatcgccgcctgccttgcccgctgctggacaggggctcggctgttgggcact
gacaattccgtggtgttgtcggggaaatcatcgtcctttccttggctgctcgcctgtgttgccacctggattctgcgcgggacgtcc
ttctgctacgtcccttcggccctcaatccagcggaccttccttcccgcggcctgctgccggctctgcggcctcttccgcgtcttcgc
cttcgccctcagacgagtcggatctccctttgggccgcctccccgcatcgataccgtcgactcgctgatcagcctcgactgtgcct
tctagttgccagccatctgttgtttgcccctcccccgtgccttccttgaccctggaaggtgccactcccactgtcctttcctaataaaa
tgaggaaattgcatcgcattgtctgagtaggtgtcattctattctggggggtggggtggggcaggacagcaagggggaggattg
ggaagacaatagcaggcatgctggggatgcggtgggctctatggcttctgaggcggaaagaaccagctggggctcgactaga
gcatggctacgtagataagtagcatgggggttaatcattaactacaaggaacccctagtgatggagttggccactccctctctgc
gcgctcgctcgctcactgaggccgggcgaccaaaggtcgcccgacgcccgggctttgcccgggcggcctcagtgagcgagc
gagcgcgcagagctttttgcaaaagcctaggcctccaaaaaagcctcctcactacttctggaatagctcagaggccgaggcgg
cctcggcctctgcataaataaaaaaaattagtcagccatggggcggagaatgggcggaactgggcggagttaggggcgggat
gggcggagttaggggcgggactatggttgctgactaattgagatgcatgctttgcatacttctgcctgctggggagcctggggac
tttccacacctggttgctgactaattgagatgcatgctttgcatacttctgcctgctggggagcctggggactttccacaccctaact
gacacacattccacagctgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaatacggttatccacagaat
caggggataacgcaggaaagaacatgtgagcaaaaggccagcaaaaggccaggaaccgtaaaaaggccgcgttgctggcg
tttttccataggctccgcccccctgacgagcatcacaaaaatcgacgctcaagtcagaggtggcgaaacccgacaggactataa
agataccaggcgtttccccctggaagctccctcgtgcgctctcctgttccgaccctgccgcttaccggatacctgtccgcctttctc
ccttcgggaagcgtggcgctttctcatagctcacgctgtaggtatctcagttcggtgtaggtcgttcgctccaagctgggctgtgtg
cacgaaccccccgttcagcccgaccgctgcgccttatccggtaactatcgtcttgagtccaacccggtaagacacgacttatcgc
cactggcagcagccactggtaacaggattagcagagcgaggtatgtaggcggtgctacagagttcttgaagtggtggcctaact
acggctacactagaagaacagtatttggtatctgcgctctgctgaagccagttaccttcggaaaaagagttggtagctcttgatcc
ggcaaacaaaccaccgctggtagcggtggtttttttgtttgcaagcagcagattacgcgcagaaaaaaaggatctcaagaagatc
ctttgatcttttctacggggtctgacgctcagtggaacgaaaactcacgttaagggattttggtcatgagattatcaaaaaggatctt
cacctagatccttttaaattaaaaatgaagttttaaatcaatctaaagtatatatgagtaaacttggtctgacagttaccaatgcttaatc
agtgaggcacctatctcagcgatctgtctatttcgttcatccatagttgcctgactccccgtcgtgtagataactacgatacgggag
ggcttaccatctggccccagtgctgcaatgataccgcgagacccacgctcaccggctccagatttatcagcaataaaccagcca
gccggaagggccgagcgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctccttcggtcctccgatcgt
tgtcagaagtaagttggccgcagtgttatcactcatggttatggcagcactgcataattctcttactgtcatgccatccgtaagatgct
tttctgtgactggtgagtactcaaccaagtcattctgagaatagtgtatgcggcgaccgagttgctcttgcccggcgtcaatacgg
gataataccgcgccacatagcagaactttaaaagtgctcatcattggaaaacgttcttcggggcgaaaactctcaaggatcttacc
gctgttgagatccagttcgatgtaacccactcgtgcacccaactgatcttcagcatcttttactttcaccagcgtttctgggtgagca
aaaacaggaaggcaaaatgccgcaaaaaagggaataagggcgacacggaaatgttgaatactcatactcttcctttttcaatatta
ttgaagcatttatcagggttattgtctcatgagcggatacatatttgaatgtatttagaaaaataaacaaataggggttccgcgcacat
ttccccgaaaagtgccacctgacgtctaagaaaccattattatcatgacattaacctataaaaataggcgtatcacgaggccctttc
gtctcgcgcgtttcggtgatgacggtgaaaacctctgacacatgcagctcccggagacggtcacagcttgtctgtaagcggatg
ccgggagcagacaagcccgtcagggcgcgtcagcgggtgttggcgggtgtcggggctggcttaactatgcggcatcagagc
agattgtactgagagtgcaccattcgacgctctcccttatgcgactcctgcattaggaagcagcccagtagtaggttgaggccgtt
gagcaccgccgccgcaaggaatggtgcatgcaaggagatggcgcccaacagtcccccggccacggggcctgccaccatac
ccacgccgaaacaagcgctcatgagcccgaagtggcgagcccgatcttccccatcggtgatgtcggcgatataggcgccagc
aaccgcacctgtggcgccggtgatgccggccacgatgcgtccggcgtagaggatctggctagcgatgaccctgctgattggtt
cgctgaccatttccgggtgcgggacggcgttaccagaaactcagaaggttcgtccaaccaaaccgactctgacggcagtttacg
agagagatgatagggtctgcttcagtaagccagatgctacacaattaggcttgtacatattgtcgttagaacgcggctacaattaat
acataaccttatgtatcatacacatacgatttaggtgacactatagaatacacggaattaattc
SEQ ID NO: 14
tagctgcgcgctcgctcgctcactgaggccgcccgggcaaagcccgggcgtcgggcgacctttggtcgcccggcctcagtga
gcgagcgagcgcgcagagagggagtggccaactccatcactaggggttccttgtagttaatgattaacccgccatgctacttatc
tacgtagccatgctctaggtacgatccccagcttgcatgcctgcaggtcactgttctcatcacatcatatcaaggttatataccatca
atattgccacagatgttacttagccttttaatatttctctaatttagtgtatatgcaatgatagttctctgatttctgagattgagtttctcat
gtgtaatgattatttagagtttctctttcatctgttcaaatttttgtctagttttattttttactgatttgtaagacttctttttataatctgcat
attacaattctctttactggggtgttgcaaatattttctgtcattctatggcctgacttttcttaatggttttttaattttaaaaataagtcttaa
tattcatgcaatctaattaacaatcttttctttgtggttaggactttgagtcataagaaatttttctctacactgaagtcatgatggcatgcttct
atattattttctaaaagatttaaagttttgccttctccatttagacttataattcactggaatttttttgtgtgtatggtatgacatatgggttc
ccttttattttttacatataaatatatttccctgtttttctaaaaaagaaaaagatcatcattttcccattgtaaaatgccatatttttttcatag
gtcacttacatatatcaatgggtctgtttctgagctctactctattttatcagcctcactgtctatccccacacatctcatgctttgctcta
aatcttgatatttagtggaacattctttcccattttgttctacaagaatatttttgttattgtctttgggctttctatatacattttgaaatgagg
ttgacaagttggggatcgtacctaatatcacaaactggaaatgtctatcaatatatagttgctctagttattaatagtaatcaattacgg
ggtcattagttcatagcccatatatggagttccgcgttacataacttacggtaaatggcccgcctggctgaccgcccaacgacccc
cgcccattgacgtcaataatgacgtatgttcccatagtaacgccaatagggactttccattgacgtcaatgggtggactatttacgg
taaactgcccacttggcagtacatcaagtgtatcatatgccaagtacgccccctattgacgtcaatgacggtaaatggcccgcctg
gcattatgcccagtacatgaccttatgggactttcctacttggcagtacatctacgtattagtcatcgctattaccatggtcgaggtga
gccccacgttctgcttcactctccccatctcccccccctccccacccccaattttgtatttatttattttttaattattttgtgcagcgatg
ggggcggggggggggggggggcgcgcgccaggcggggcggggcggggcgaggggcggggcggggcgaggcggag
aggtgcggcggcagccaatcagagcggcgcgctccgaaagtttccttttatggcgaggcggcggcggcggcggccctataaa
aagcgaagcgcgcggggggggagtcgctgcgacgctgccttcgccccgtgccccgctccgccgccgcctcgcgccgcc
cgccccggctctgactgaccgcgttactcccacaggtgagcggggggacggcccttctcctccgggctgtaattagcgcttgg
tttaatgacggcttgtttcttttctgtggctgcgtgaaagccttgaggggctccgggagggccctttgtgcggggggagcggctcg
gggctgtccgcggggggacggctgccttcgggggggacggggcagggggggttcggcttctggcgtgtgaccggcggct
ctagagcctctgctaaccatgttcatgccttcttctttttcctacagctcctgggcaacgtgctggttattgtgctgtctcatcattttgg
caaagaattggatccactcgagttagccatgtacggcaggaagaagaggaggcagaggaggagggccgtggtgaccggcgt
gaccgccgtggccgagagatcttatccgtatgatgttcctgattatgcttgatagtaagcttatcgataatcaacctctggattacaa
aatttgtgaaagattgactggtattcttaactatgttgctccttttacgctatgtggatacgctgctttaatgcctttgtatcatgctattgc
ttcccgtatggctttcattttctcctccttgtataaatcctggttgctgtctctttatgaggagttgtggcccgttgtcaggcaacgtggc
gtggtgtgcactgtgtttgctgacgcaacccccactggttggggcattgccaccacctgtcagctcctttccgggactttcgctttc
cccctccctattgccacggcggaactcatcgccgcctgccttgcccgctgctggacaggggctcggctgttgggcactgacaat
tccgtggtgttgtcggggaaatcatcgtcctttccttggctgctcgcctgtgttgccacctggattctgcgcgggacgtccttctgct
acgtcccttcggccctcaatccagcggaccttccttcccgcggcctgctgccggctctgcggcctcttccgcgtcttcgccttcgc
cctcagacgagtcggatctccctttgggccgcctccccgcatcgataccgtcgactcgctgatcagcctcgactgtgccttctagt
tgccagccatctgttgtttgcccctcccccgtgccttccttgaccctggaaggtgccactcccactgtcctttcctaataaaatgagg
aaattgcatcgcattgtctgagtaggtgtcattctattctggggggtggggtggggcaggacagcaagggggaggattgggaag
acaatagcaggcatgctggggatgcggtgggctctatggcttctgaggcggaaagaaccagctggggctcgactagagcatg
gctacgtagataagtagcatgggggttaatcattaactacaaggaacccctagtgatggagttggccactccctctctgcgcgct
cgctcgctcactgaggccgggcgaccaaaggtcgcccgacgcccgggctttgcccgggcggcctcagtgagcgagcgagc
gcgcagagctttttgcaaaagcctaggcctccaaaaaagcctcctcactacttctggaatagctcagaggccgaggcggcctcg
gcctctgcataaataaaaaaaattagtcagccatggggcggagaatgggcggaactgggcggagttaggggcgggatgggc
ggagttaggggcgggactatggttgctgactaattgagatgcatgctttgcatacttctgcctgctggggagcctggggactttcc
acacctggttgctgactaattgagatgcatgctttgcatacttctgcctgctggggagcctggggactttccacaccctaactgaca
cacattccacagctgcattaatgaatcggccaacgcgcggggagaggcggtttgcgtattgggcgctcttccgcttcctcgctca
ctgactcgctgcgctcggtcgttcggctgcggcgagcggtatcagctcactcaaaggcggtaatacggttatccacagaatcag
gggataacgcaggaaagaacatgtgagcaaaaggccagcaaaaggccaggaaccgtaaaaaggccgcgttgctggcgttttt
ccataggctccgcccccctgacgagcatcacaaaaatcgacgctcaagtcagaggtggcgaaacccgacaggactataaaga
taccaggcgtttccccctggaagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctccaagctgggctgtgtgcac
gaaccccccgttcagcccgaccgctgcgccttatccggtaactatcgtcttgagtccaacccggtaagacacgacttatcgccac
tggcagcagccactggtaacaggattagcagagcgaggtatgtaggcggtgctacagagttcttgaagtggtggcctaactacg
gctacactagaagaacagtatttggtatctgcgctctgctgaagccagttaccttcggaaaaagagttggtagctcttgatccggc
aaacaaaccaccgctggtagcggtggtttttttgtttgcaagcagcagattacgcgcagaaaaaaaggatctcaagaagatccttt
gatcttttctacggggtctgacgctcagtggaacgaaaactcacgttaagggattttggtcatgagattatcaaaaaggatcttcac
ctagatccttttaaattaaaaatgaagttttaaatcaatctaaagtatatatgagtaaacttggtctgacagttaccaatgcttaatcagt
gaggcacctatctcagcgatctgtctatttcgttcatccatagttgcctgactccccgtcgtgtagataactacgatacgggagggc
ttaccatctggccccagtgctgcaatgataccgcgagacccacgctcaccggctccagatttatcagcaataaaccagccagcc
ggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagc
tccggttcccaacgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatccgtaagatgcttttc
tgtgactggtgagtactcaaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactctcaaggatcttaccgctg
ttgagatccagttcgatgtaacccactcgtgcacccaactgatcttcagcatcttttactttcaccagcgtttctgggtgagcaaaaa
caggaaggcaaaatgccgcaaaaaagggaataagggcgacacggaaatgttgaatactcatactcttcctttttcaatattattga
agcatttatcagggttattgtctcatgagcggatacatatttgaatgtatttagaaaaataaacaaataggggttccgcgcacatttcc
ccgaaaagtgccacctgacgtctaagaaaccattattatcatgacattaacctataaaaataggcgtatcacgaggccctttcgtct
cgcgcgtttcggtgatgacggtgaaaacctctgacacatgcagctcccggagacggtcacagcttgtctgtaagcggatgccgg
gagcagacaagcccgtcagggcgcgtcagcgggtgttggcgggtgtcggggctggcttaactatgcggcatcgagcagatt
gtactgagagtgcaccattcgacgctctcccttatgcgactcctgcattaggaagcagcccagtagtaggttgaggccgttgagc
accgccgccgcaaggaatggtgcatgcaaggagatggcgcccaacagtcccccggccacggggcctgccaccatacccac
gccgaaacaagcgctcatgagcccgaagtggcgagcccgatcttccccatcggtgatgtcggcgatataggcgccagcaacc
gcacctgtggcgccggtgatgccggccacgatgcgtccggcgtagaggatctggctagcgatgaccctgctgattggttcgct
gaccatttccgggtgcgggacggcgttaccagaaactcagaaggttcgtccaaccaaaccgactctgacggcagtttacgaga
gagatgatagggtctgcttcagtaagccagatgctacacaattaggcttgtacatattgtcgttagaacgcggctacaattaataca
taaccttatgtatcatacacatacgatttaggtgacactatagaatacacggaattaattc
SEQ ID NO: 15
GRKKRRQRRRPQ
SEQ ID NO: 16
YPYDVPDYA
SEQ ID NO: 17
KKKKKK
SEQ ID NO: 18
HHHHHH
SEQ ID NO: 19
GKPIPNPLLGLDST
SEQ ID NO: 20
EQKLISEEDL
SEQ ID NO: 21
DYKDDDDK

### OTHER EMBODIMENTS

It is to be understood that while the disclosure has been described in conjunction with the detailed description thereof, the foregoing description is intended to illustrate and not limit the scope, which is defined by the appended claims. Other aspects, advantages, and modifications are within the scope of the following claims.

1. A composition comprising a viral vector, wherein the viral vector comprises a coding sequence that encodes a peptide comprising the amino acid sequence:
(SEQ ID NO: 1)
AVVWGVTAV
or
(SEQ ID NO: 2)
AVVTGVTAV.

2. The composition of claim 1, wherein the viral vector comprises a coding sequence that encodes a peptide comprising the amino acid sequence:
(SEQ ID NO: 3)
GAVVWGVTAVKK
or
(SEQ ID NO: 4)
RAVVTGVTAVAE.

3. The composition of claim 1, wherein the viral vector comprises a coding sequence that encodes a peptide comprising the amino acid sequence:
(SEQ ID NO: 6)
GAVVWGVTAVKKGRKKRRQRRRPQ
or
(SEQ ID NO: 7)
YGRKKRRQRRRAVVTGVTAVAE.

4. The composition of claim 1, wherein the peptide is from 9 to 45 amino acids in length.

5.-7. (canceled)

8. The composition of claim 1, wherein the peptide inhibits α-synuclein (SEQ ID NO: 8) aggregation by binding to residues 68-78 of α-synuclein.

9. The composition of claim 1, wherein the viral vector is an AAV vector.

10. The composition of claim 9, wherein the AAV vector is an AAV serotype 1 vector (AAV1), an AAV serotype 2 vector (AAV2), an AAV serotype 1 and 2 hybrid vector (AAV1/2), an AAV serotype 3 vector (AAV3), an AAV serotype 4 vector (AAV4), an AAV serotype 5 vector (AAV5), an AAV serotype 6 vector (AAV6), an AAV serotype 7 vector (AAV7), an AAV serotype 8 vector (AAV8), or an AAV serotype 9 vector (AAV9).

11.-12. (canceled)

13. The composition of claim 1, wherein the viral vector comprises a feature selected from a coding sequence that encodes a heterologous peptide tag, a regulatory sequence, a post-transcriptional regulatory element, a scaffold attachment region sequence, and a polyadenylation signal sequence.

14. (canceled)

15. The composition of claim 13, wherein the heterologous peptide tag is a human influenza hemagglutinin (HA) tag.

16. (canceled)

17. The composition of claim 13, wherein regulatory sequence comprises a promoter.

18.-20. (canceled)

21. The composition of claim 13, wherein the regulatory sequence is a hybrid CMV enhancer/CBA promoter.

22.-30. (canceled)

31. The composition of claim 1, wherein the viral vector comprises a nucleic acid sequence of SEQ ID NO: 13 or SEQ ID NO: 14.

32.-64. (canceled)

65. A method for treating a neurodegenerative disease in a subject, the method comprising administering to the subject an effective amount of the composition of claim 1.

66. The method of claim 65, wherein the neurodegenerative disease is Alzheimer's disease or a synucleinopathy.

67. The method of claim 66, wherein the synucleinopathy is selected from the group consisting of: a Lewy body disorder, Parkinson's disease (PD), PD with dementia (PDD), pure autonomic failure (PAF), and multiple system atrophy (MSA).

68. A method for reducing α-synuclein (SEQ ID NO: 8) aggregation in a subject in need thereof, the method comprising administering to the subject an effective amount of the composition of claim 1.

69. The method of claim 68, wherein the subject has a neurodegenerative disease.

70. The method of claim 69, wherein the neurodegenerative disease is Alzheimer's disease or a synucleinopathy.

71. The method of claim 70, wherein the synucleinopathy is a Lewy body disorder, Parkinson's disease (PD), PD with dementia (PDD), pure autonomic failure (PAF), or multiple system atrophy (MSA).

72.-75. (canceled)

76. A method of expressing a peptide that inhibits α-synuclein (SEQ ID NO: 8) aggregation in a mammalian cell, the method comprising introducing the viral vector of claim 1 into the mammalian cell.

77.-78. (canceled)

79. A kit comprising:
(i) the composition of claim 1; and
(ii) instructions for performing the method of claim 65.
